(19) United States
(12) Patent Application Publication
(10) Pub. No.: US 2026/0157665 A1
(43) Pub. Date: Jun. 11, 2026

(54) SYSTEM AND METHOD FOR MONITORING ANALYTES WITHIN BLOOD, BODILY FLUIDS, OR TISSUE OF A PATIENT

(71) Applicant: CloverSense LLC, Manassas, VA (US)

(72) Inventors: Jacob D. Conner, Rogersville, MO (US); Daniel A. Bartholomeusz, Poway, CA (US); John F. Tichenor, San Diego, CA (US)

(21) Appl. No.: 18/975,411

(22) Filed: Dec. 10, 2024

(51) Int. Cl. A61B 5/145

(52) U.S. Cl. CPC A61B 5/14546; A61B 5/14532; A61B 5/1455

(57) ABSTRACT

An analyte sensing system (210) includes a sensor assembly (216) and a sensor (214). The sensor assembly (216) includes an energy source (228) that generates energy. The sensor (214) includes (i) an energy guide (218) that receives the energy from the energy source (228), (ii) a sheath (220) coupled to the energy guide (218) near a guide distal end (218D), the sheath (220) being substantially cylindrical-shaped to define at least a portion of a reaction chamber (222) therewithin that extends distally away from the guide distal end (218D), the sheath (220) being oxygen permeable and analyte impermeable, (iii) a sensing polymer (224) positioned near the guide distal end (218D) of the energy guide (218), the energy guide (218) guiding the energy from the energy source (228) toward the sensing polymer (224), the sensing polymer (224) being configured to sense one of oxygen and the analyte, and (iv) a transduction matrix (226) retained substantially within the reaction chamber (222). The oxygen and the analyte follow different diffusion paths within the transduction matrix (226).

### BACKGROUND

Diabetes is an increasingly relevant health issue for millions of people throughout the world. The International Diabetes Federation (IDF) estimates that there were 537 million adults (aged 20-79) living with diabetes in 2021, and this number is expected to increase to 642 million by 2040. The IDF also reports that the prevalence of diabetes is growing globally, with the highest increase witnessed in low-and middle-income countries.

Factors such as aging, obesity, and unhealthy lifestyles have been found to contribute to the prevalence of diabetes, with obesity being a known major factor contributing to diabetes. According to the World Health Organization (WHO), in 2022, the number of obese individuals worldwide exceeded one billion, including 650 million adults, 340 million adolescents, and 39 million children. This number continues to grow. If things continue as they are, the WHO predicts that by 2025, around 167 million people, both adults and children, will experience worsening health problems due to their weight issues.

Due to the increase in the prevalence of diabetes in the global population, there has been a corresponding increase in the prescription of a medical device known as a Continuous Glucose Monitor (CGM) to help diabetics monitor and indirectly or directly control blood glucose levels. In particular, the use of CGM devices is projected to grow at a compound annual growth rate (CAGR) of 7.19% from 2024 to 2030.

Unfortunately, trust in CGM use among diabetics has not significantly improved over the last decade due to limitations of the sensor accuracy. In particular, lack of trust in CGM devices is due, at least in part, to evidence that these CGM devices are subject to sporadic, unpredictable, large errors. For example, the FDA Manufacturer and User Facility Device Experience (MAUDE) was established as a surveillance tool for monitoring case reports of problems and safety issues with such devices. A text analysis of reports to the FDA MAUDE database since 2015 reveals over 25,000 complaints of CGM sensor inaccuracy in comparison to more accurate Blood Glucose Monitor (BGM) readings, with many instances directly leading to serious outcomes. Approximately 55 percent of reported differences between concurrent CGM and BGM readings show differences of 100 mg/dl or more. For the year 2022, CGM devices had a total of 281,963 adverse events with 268,310 malfunctions, 13,644 injuries, and nine deaths (as the manufacturer comments on each event, the total number of records was 583,321).

Some currently available CGM devices utilize hydrogen peroxide probes for sensing the level of glucose within the blood of a patient. However, hydrogen peroxide probes encounter significant challenges due to electrochemical interference in complex matrices like the body. This interference causes errors in measurements by oxidizing other electroactive constituents along with hydrogen peroxide, leading to variable and positive net errors. Additionally, hydrogen peroxide can react undesirably with surrounding tissue and degrade the enzyme needed for sensor operation. Even with glucose oxidase coupled to a transducer, issues persist if oxygen is not in excess, particularly in subcutaneous tissue where oxygen levels fluctuate. These problems necessitate addressing background oxygen variations for accurate measurements. Despite efforts to stabilize electrodes and minimize electroactive interference, challenges remain. Alternative optical methods are desirable, especially in oxygen-scarce environments, but they also require addressing selective oxygen sensing issues.

Further, electrochemical sensors, including those used in CGM devices, face several challenges when deployed within the body. Some of the common issues include (i) low limit of detection (achieving a low level of detection is crucial for detecting low concentrations of analytes, which is often required for early disease diagnosis), (ii) non-specific adsorption (suppressing the non-specific adsorption of interfering species is necessary to avoid false readings and maintain sensor accuracy), (iii) reproducibility and stability (ensuring consistent performance over time and in different conditions is challenging especially in the complex environment of the body), (iv) biofouling (the accumulation of biological material on the sensor surface can interfere with sensor function and lead to inaccurate readings), (v) fibrous encapsulation (the body's response to a foreign object can lead to encapsulation of the sensor, thereby impairing its function), and (vi) inflammation and loss of host vasculature (the body's immune response can cause inflammation around the sensor, affecting its accuracy and leading to potential complications). For CGM sensors, specifically, issues such as sensor calibration, lifetime, and the need for frequent replacement due to biofouling or sensor drift are important concerns.

Moreover, other previously used sensors have also experienced certain problems such as similar issues relating to implanted electronics, and low optical coupling efficiency.

Accordingly, it is desired to develop a system and method for sensing analytes, such as glucose, within blood, other bodily fluids, and/or tissue of a patient in a manner that is reliable, safe, and cost-efficient, and that overcomes the various drawbacks noted within currently available CGM devices, thereby helping diabetics manage their glucose levels by improving sensor accuracy and reliability.

### SUMMARY

The present invention is directed toward an analyte sensing system for sensing an analyte within blood, other bodily fluids (sweat, urine, tears, saliva etc.), or tissue of a patient (which can be in-vivo and/or extracorporeal). In various embodiments, the analyte sensing system includes a sensor assembly and a sensor. The sensor assembly includes an energy source that generates energy. The sensor includes (i) an energy guide that receives the energy from the energy source, the energy guide including a guide distal end, (ii) a sheath that is coupled to the energy guide near the guide distal end, the sheath being substantially cylindrical-shaped to define at least a portion of a reaction chamber therewithin that extends distally away from the guide distal end, the sheath being oxygen permeable, and the sheath being impermeable to the analyte being sensed, the sheath having a sheath distal end, (iii) a sensing polymer that is positioned near the guide distal end of the energy guide, the energy guide guiding the energy from the energy source toward the sensing polymer, the sensing polymer being configured to sense one of oxygen and the analyte, and (iv) a transduction matrix that is retained substantially within the reaction chamber. In many embodiments, the oxygen that permeates through the sheath and into the transduction matrix that is retained within the reaction chamber follows a first diffusion path within the transduction matrix; and the analyte permeates into the transduction matrix that is retained within the reaction chamber through the sheath distal end of the sheath, the analyte following a second diffusion path within the transduction matrix that is different than the first diffusion path.

In some embodiments, the sensing polymer is coated onto the guide distal end of the energy guide.

In certain embodiments, the sensing polymer defines a chamber proximal end of the reaction chamber.

In some embodiments, the sensing polymer is hydrophobic, and the transduction matrix is hydrophilic.

In many embodiments, the sensing polymer is an oxygen sensing polymer that is configured to sense the oxygen within the transduction matrix.

In other embodiments, the sensing polymer is configured to directly sense the analyte within the transduction matrix.

In some embodiments, the sheath is formed via one of a three-dimensional extrusion technique and a three-dimensional molding technique.

In many embodiments, the sheath has a concentric unibody design.

In certain embodiments, the substantially cylindrical shape of the sheath defines a chamber diameter of the reaction chamber, the chamber diameter being between approximately 100 nanometers (nm) and 500 micrometers (μm).

In some embodiments, the sheath is formed from one or more of fluorinated ethylene propylene (FEP), paraformaldehyde (PFA), polytetrafluoroethylene (PTFE), polyimide, polyether block amide (PEBA), polyvinylchloride (PVC), polydimethylsiloxane, polyurethane, polyethylene, polycarbonate, poly(1-trimethylsilyl-1-propyne) (PTMSP), ethylene vinyl alcohol (EVOH), sulfonated tetrafluoroethylene-based fluoropolymer-copolymer, and modified cellulose.

In one embodiment, the sheath is formed at least partially from fluorinated ethylene propylene (FEP).

In many embodiments, the transduction matrix is comprised of a hydrogel and one or more enzymes that are configured to react with the oxygen and the analyte.

In certain embodiments, a reaction between the one or more enzymes with the oxygen and the analyte consumes at least a portion of the oxygen and the analyte that is present within the transduction matrix.

In some embodiments, the transduction matrix further includes a catalyst that is configured to initiate a reaction between the one or more enzymes with the oxygen and the analyte.

In certain embodiments, the sheath has a wall thickness that impacts the permeability of the sheath to oxygen; and wherein the wall thickness of the sheath is between approximately 10 micrometers and 400 micrometers.

In some embodiments, the sheath has an oxygen permeability of between approximately five (cm³ mm/m²·day·bar)/mm and 60,000 (cm³ mm/m²·day·bar)/mm.

In certain embodiments, the sensor further includes a buffer layer that is positioned about the energy guide, the buffer layer being formed from one or more polymeric materials.

In some embodiments, the energy source is a light source that generates light energy, and the energy guide is an optical fiber.

In certain embodiments, the sensor is a first sensor channel.

In some embodiments, the analyte sensing system further includes a second sensing channel that includes (i) a second energy guide that receives the energy from the energy source, the second energy guide including a second guide distal end, (ii) a second sheath that is coupled to the second energy guide near the second guide distal end, the second sheath defining at least a portion of a second reaction chamber, the second sheath being oxygen permeable, and the second sheath being impermeable to the analyte being sensed, (iii) a second sensing polymer that is positioned near the second guide distal end of the second energy guide, the second energy guide guiding the energy from the energy source toward the second sensing polymer, the second sensing polymer being configured to sense one of the oxygen and the analyte, and (iv) a second transduction matrix that is retained substantially within the second reaction chamber.

In certain embodiments, the analyte sensing system further includes a reference channel that includes (i) a reference energy guide that receives the energy from the energy source, the reference energy guide including a guide distal end, (ii) a reference sheath that is coupled to the reference energy guide near the guide distal end, and (iii) a reference sensing polymer that is configured to sense oxygen from within the blood, bodily fluids or tissue of the patient to set a baseline level of oxygen within the blood, bodily fluids or tissue of the patient.

The present invention is further directed toward an analyte sensing system for sensing an analyte within blood, bodily fluids or tissue of a patient, the analyte sensing system including a sensor assembly including an energy source that generates energy; and a sensor including (i) an energy guide that receives the energy from the energy source, the energy guide including a guide distal end, (ii) a sheath that is coupled to the energy guide near the guide distal end, the sheath having a concentric unibody design that is substantially cylindrical-shaped to define at least a portion of a reaction chamber therewithin that extends distally away from the guide distal end, the sheath being oxygen permeable, and the sheath being impermeable to the analyte being sensed, the sheath having a sheath distal end, (iii) a hydrophobic oxygen sensing polymer that is coated onto the guide distal end of the energy guide, the oxygen sensing polymer being configured to sense oxygen within the reaction chamber, the oxygen sensing polymer defining a chamber proximal end of the reaction chamber, the energy guide guiding the energy from the energy source toward the oxygen sensing polymer, and (iv) a hydrophilic transduction matrix that is retained substantially within the reaction chamber, the transduction matrix including a hydrogel and one or more enzymes that are configured to react with the oxygen and the analyte; wherein the oxygen that permeates through the sheath and into the transduction matrix that is retained within the reaction chamber follows a first diffusion path within the transduction matrix; wherein the analyte permeates into the transduction matrix that is retained within the reaction chamber through the sheath distal end of the sheath, the analyte following a second diffusion path within the transduction matrix that is different than the first diffusion path; and wherein a reaction between the one or more enzymes with the oxygen and the analyte consumes at least a portion of the oxygen and the analyte that is present within the transduction matrix.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic illustration of an analyte sensing system having features of the present invention that is positioned to extend at least partially within a body of a patient, the analyte sensing system including a sensor, and a sensor assembly;

FIG. 2A is a simplified schematic cutaway view illustration of an embodiment of the analyte sensing system illustrated in FIG. 1, including an embodiment of the sensor that includes at least an energy guide, a sheath, a sensing polymer, and a transduction matrix;

FIG. 2B is a sectional view illustration of the analyte sensing system taken on line 2B-2B in FIG. 2A;

FIG. 2C is a sectional view illustration of the analyte sensing system taken on line 2C-2C in FIG. 2A;

FIG. 3A is a representative illustration showing a theoretical analyte profile that may be realized during use of the analyte sensing system illustrated in FIG. 2A;

FIG. 3B is a representative illustration showing a theoretical oxygen profile that may be realized during use of the analyte sensing system illustrated in FIG. 2A;

FIG. 4A is a simplified schematic illustration of another embodiment of the sensor that can be included as part of the analyte sensing system;

FIG. 4B is a sectional view illustration of the sensor taken on line 4B-4B in FIG. 4A;

FIG. 5A is a simplified schematic illustration of still another embodiment of the sensor that can be included as part of the analyte sensing system;

FIG. 5B is a sectional view illustration of the sensor taken on line 5B-5B in FIG. 5A;

FIG. 6A is a simplified schematic illustration of another embodiment of the sensor that can be included as part of the analyte sensing system;

FIG. 6B is a sectional view illustration of the sensor taken on line 6B-6B in FIG. 6A;

FIG. 7A is a simplified schematic illustration of yet another embodiment of the sensor that can be included as part of the analyte sensing system;

FIG. 7B is a sectional view illustration of the sensor taken on line 7B-7B in FIG. 7A;

FIG. 8A is a simplified schematic illustration of another embodiment of the sensor that can be included as part of the analyte sensing system;

FIG. 8B is a sectional view illustration of the sensor taken on line 8B-8B in FIG. 8A;

FIG. 8C is a sectional view illustration of the sensor taken on line 8C-8C in FIG. 8A;

FIG. 9A is a simplified schematic illustration of still yet another embodiment of the sensor that can be included as part of the analyte sensing system;

FIG. 9B is a sectional view illustration of the sensor taken on line 9B-9B in FIG. 9A;

FIG. 9C is a sectional view illustration of the sensor taken on line 9C-9C in FIG. 9A;

FIG. 10 is a simplified schematic illustration of another embodiment of the analyte sensing system illustrated in FIG. 1, including still another embodiment of the sensor, where the sensing polymer is provided in an alternative format;

FIG. 11A is a simplified schematic cutaway view illustration of still another embodiment of the analyte sensing system illustrated in FIG. 1, including still another embodiment of the sensor that further includes a protective buffer layer that is positioned about the energy guide;

FIG. 11B is a sectional view illustration of the analyte sensing system taken on line 11B-11B in FIG. 11A;

FIG. 12 is a simplified schematic illustration of yet another embodiment of the sensor that can be included as part of the analyte sensing system;

FIG. 13A is a simplified schematic illustration of still another embodiment of the sensor that can be included as part of the analyte sensing system;

FIG. 13B is a sectional view illustration of the sensor taken on line 13B-13B in FIG. 13A;

FIG. 13C is a sectional view illustration of the sensor taken on line 13C-13C in FIG. 13A;

FIG. 14A is a simplified schematic illustration of still another embodiment of the sensor that can be included as part of the analyte sensing system;

FIG. 14B is a sectional view illustration of the sensor taken on line 14B-14B in FIG. 14A;

FIG. 14C is a sectional view illustration of the sensor taken on line 14C-14C in FIG. 14A;

FIG. 15A is a simplified schematic illustration of still another embodiment of the sensor that can be included as part of the analyte sensing system;

FIG. 15B is a sectional view illustration of the sensor taken on line 15B-15B in FIG. 15A;

FIG. 15C is a sectional view illustration of the sensor taken on line 15C-15C in FIG. 15A;

FIG. 16 is a simplified schematic illustration of another embodiment of the sensor that can be included as part of the analyte sensing system, with the sensor being configured without the energy guide;

FIG. 17 is a simplified schematic illustration of still another embodiment of the sensor that can be included as part of the analyte sensing system, with the sensor again being configured without the energy guide, but with the sensor further including an energy transmission facilitator;

FIG. 18 is a simplified schematic illustration of yet another embodiment of the sensor that can be included as part of the analyte sensing system, with the sensor yet again being configured without the energy guide; and

FIG. 19 is a simplified schematic illustration of still yet another embodiment of the sensor that can be included as part of the analyte sensing system, with the sensor including multiple energy guides.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Embodiments of the present invention are described herein in the context of a system and method for continuously monitoring analytes, such as glucose, lactates, ketones, cholesterol, bilirubin, alcohol, pyruvate, oxalates, xanthene, NADPH, cytochrome c, electrolytes, allergens, histamines, etc., within blood, other bodily fluids, and/or tissue of a patient. More particularly, the present invention is directed toward a sensor that is configured to continuously, safely, accurately, and reliably sense the amount of an analyte of interest within the blood, bodily fluids, and/or tissue of the patient in a cost-efficient manner that overcomes various drawbacks found within currently available devices. In some embodiments, the present invention encompasses a multi-channel sensor, having any suitable number of sensor channels, which allows for an enhanced level of quality assurance by providing near real time self-calibration and site health monitoring. As described herein, in certain embodiments, the sensor can have an opto-enzymatic design that is nearly impervious to most medication reactions, and many antioxidant foods can be adjusted for based on inclusion of a built-in oxygen reference channel.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same or similar reference indicators will be used throughout the drawings and the following detailed description to refer to the same or like parts.

In the interest of clarity, not all of the routine features of the implementations described herein are shown and described. It will, of course, be appreciated that in the development of any such actual implementations, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

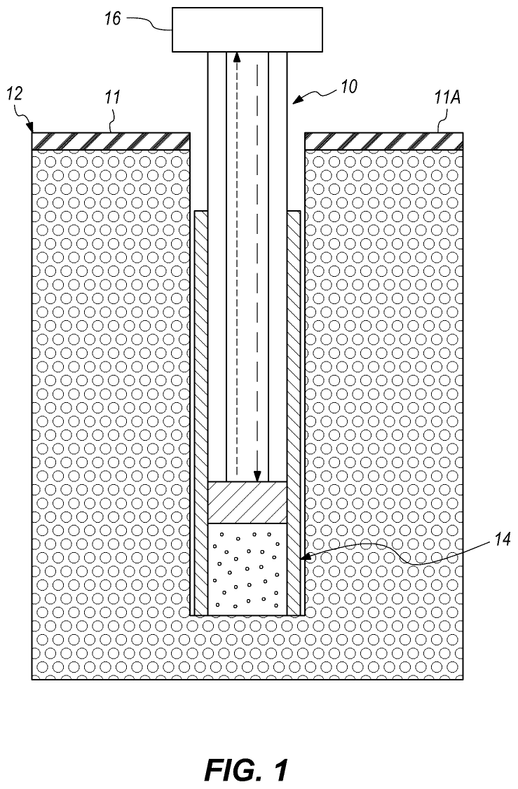

FIG. 1 is a simplified schematic illustration of an analyte sensing system 10 having features of the present invention that can be positioned to extend at least partially within a body 11 of a patient 12, such as beneath the epidermis 11A of the body 11 of the patient 12. In this manner, the analyte sensing system 10 can be utilized to sense an analyte of interest within the body 11 of the patient 12 in vivo. Alternatively, the analyte sensing system 10 can be utilized to sense the analyte of interest in vitro or extracorporeal, such as within sweat, tears, urine, saliva, etc.

In various embodiments, the analyte sensing system 10 can include a sensor 14 that is configured to sense an analyte, directly or indirectly, within blood, bodily fluids, and/or tissue of the patient 12, and a sensor assembly 16 that receives a signal from the sensor 14 regarding the sensed analyte to determine a level, or volume, of the analyte within the blood, bodily fluids, and/or tissue of the patient 12. In many embodiments illustrated and described herein, the analyte sensing system 10 is configured to indirectly determine the level of the analyte of interest within the blood, bodily fluids, and/or tissue of the patient 12. For example, in some embodiments, the analyte sensing system 10 is configured to directly sense and/or determine a level of oxygen remaining after the oxygen and the analyte of interest react with an enzyme within the sensor 14. In such embodiments, the level of oxygen remaining is representative of the level of the analyte of interest that is present within the blood, bodily fluids, and/or tissue of the patient 12. Alternatively, in other embodiments, the analyte sensing system 10 can be configured to sense and/or determine a pH level within the sensor 14, with the pH level again being representative of the level of the analyte of interest that is present within the blood, bodily fluids, and/or tissue of the patient 12. This can be accomplished by measuring the production of an end product, for example gluconic acid, lactic acid etc., as opposed to measuring the concentration or partial pressure of a reactant (such as glucose, lactate, O₂, etc.). Still alternatively, the analyte sensing system 10 can be configured to sense and/or determine, directly or indirectly, the analyte of interest. Yet alternatively, the analyte sensing system 10 and/or the sensor 14 can be configured to directly or indirectly sense and/or determine a level of the analyte of interest within the blood, or bodily fluids, interstitial fluid, urine, sweat, tears, saliva, and/or tissue of the patient 12.

In certain embodiments, the analyte sensing system 10 and/or the sensor 14 is described as being useful for determining the level of glucose within the blood, bodily fluids, and/or tissue of the patient 12. However, in other embodiments, the analyte sensing system 10 and/or the sensor 14 can be utilized to determine the level of other analytes, such as lactates, ketones, cholesterol, bilirubin, NADPH, oxalates, citrates, alcohol, pyruvates, cytochromes, or xanthenes, etc., within the blood, bodily fluids, and/or tissue of the patient 12. Thus, the description of the present invention being particularly useful in sensing and/or determining the level of glucose in the blood, bodily fluids, and/or tissue of the patient 12 is not intended to be limiting in any manner.

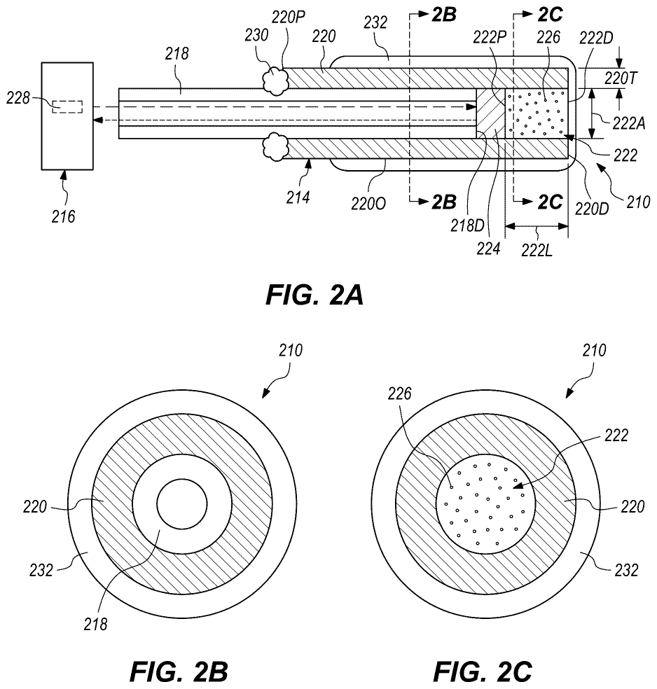

FIG. 2A is a simplified schematic cutaway view illustration of an embodiment of the analyte sensing system 210 illustrated in FIG. 1. As illustrated, in various embodiments, the analyte sensing system 210 includes a sensor 214 that is configured to sense an analyte, such as glucose or another suitable analyte, within the blood, bodily fluids or tissue of the patient 12 (illustrated in FIG. 1), and a sensor assembly 216 (illustrated as a box) that receives a signal from the sensor 214 regarding the sensed analyte to determine a level (or volume) of the analyte within the blood, bodily fluids or tissue of the patient 12. As illustrated, the sensor 214 in this embodiment is a single channel sensor. Alternatively, in many embodiments, the sensor 214 can be a multi-channel sensor, with each sensor channel having a similar overall design. Still alternatively, in some embodiments, the sensor 214 may be a multi-channel sensor having a plurality of waveform transmission channels and a single sheath comprising a unibody construction and containing one transduction matrix. In such embodiments, the multiple sensing elements can be positioned at substantially the same longitudinal position for redundancy, or the multiple sensing elements can be staggered to provide different sensing ranges. It is appreciated that such multi-channel sensors can have any suitable number of sensor channels to provide enhanced accuracy and reliability regardless of the environment in which the sensor 214 is being used. For example, in certain non-exclusive alternative embodiments, the sensor 214 can include 2, 3, 4, 5, 6, 7, 8, 9, 10, 11, 12, 13, 14, 15, 16, 17, 18, 19, or 20 individual sensor channels.

As noted above, it is appreciated that the sensor 214 and/or the analyte sensing system 210 can directly or indirectly sense the analyte in order to effectively and accurately determine the level of the analyte within the blood, bodily fluids or tissue of the patient 12.

In many embodiments, the sensor 214 can include one or more of (i) an energy guide 218, (ii) a sheath 220 that defines at least a portion of a reaction chamber 222, (iii) a sensing polymer 224, and (iv) a transduction matrix 226 that can be received and retained substantially, if not entirely, within the reaction chamber 222.

In various embodiments, the sensor assembly 216 can include an energy source 228 (illustrated as a box in phantom) that is configured to generate energy that is guided by and/or directed through the energy guide 218 of the sensor 214 toward the sensing polymer 224 and/or the transduction matrix 226. It is appreciated that the energy source 228 can be configured to generate any suitable type of energy that is guided by and/or directed through the energy guide 218 of the sensor 214 toward the sensing polymer 224 and/or the transduction matrix 226. For example, in certain non-exclusive embodiments, the energy source 228 can be a light source that generates light energy that is then guided by and/or directed through the energy guide 218 of the sensor 214 toward the sensing polymer 224 and/or the transduction matrix 226. In such embodiments, the energy guide 218 will typically be provided in the form of an optical fiber. Alternatively, the energy source 228 can be configured to generate electrical energy, chemical energy, or another suitable type of energy that is then guided by and/or directed through the energy guide 218 of the sensor 214 toward the sensing polymer 224 and/or the transduction matrix 226. In such alternative embodiments, the energy guide 218 will have a design that is particularly suitable for guiding the specific type of energy therethrough.

Still alternatively, the analyte sensing system 210, the sensor 214 and/or the sensor assembly 216 can include more components or fewer components than what is specifically illustrated and described in relation to FIG. 2A.

As an overview, in various embodiments, the present invention is directed toward the design of the sensor 214 and its corresponding method of operation. In many such embodiments, the sensor 214 can be generally described as an oxygen-permeable sheath sensor that is intended to be a disposable portion of the invention that is injected into an organism, such as the body 11 (illustrated in FIG. 1) of the patient 12, to detect glucose, or another suitable analyte of interest, therein. For example, the sheath 220 can be a three-dimensional, tubular-shaped or cylindrical-shaped sheath that is oxygen permeable, while also being impermeable to the analyte of interest, such that oxygen is allowed to permeate and diffuse from the blood, bodily fluids, and/or tissue of the patient 12 through the structure of the sheath 220, and into and through the transduction matrix 226 retained within the reaction chamber 222. Alternatively, the sheath 220 can have another suitable shape and/or design, such as being substantially elliptical-shaped, triangular-shaped, or square-shaped, or being a multi-lumen type extrusion sheath.

As referred to herein, the sheath 220 being “impermeable” to the analyte of interest is intended to signify that the sheath 220 is designed to allow very little or no analyte of interest to permeate therethrough. Stated in another manner, although the sheath being “impermeable” to the analyte of interest can include the sheath 220 being fully impermeable to the analyte of interest, in some embodiments, the sheath 220 can be “leaky” so as to still conduct a small amount of the analyte of interest (such as glucose).

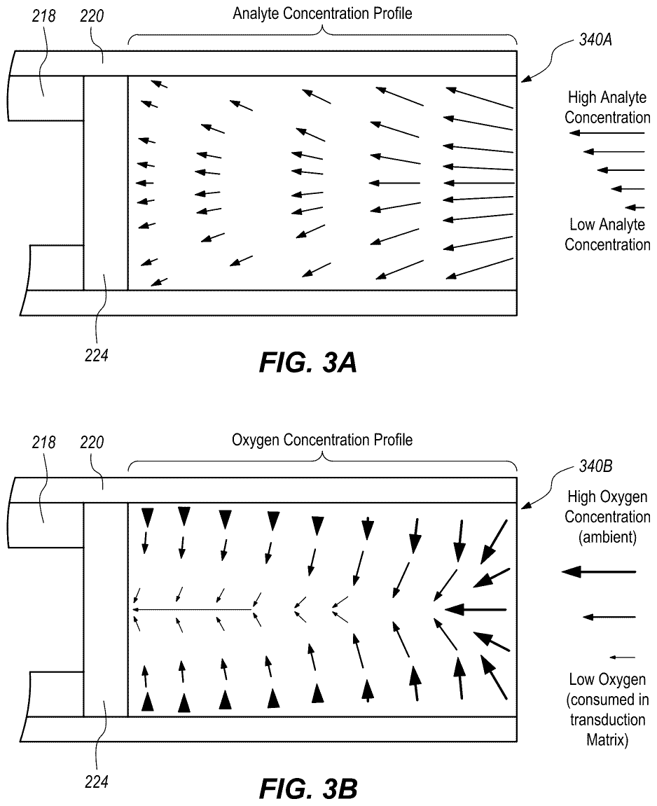

It is appreciated that the specific level of oxygen permeability, and thus the related diffusion rate, can be influenced by various design features that are incorporated into the sheath 220. At the same time, a sheath distal end 220D of the sheath 220, which can be open in certain embodiments, can allow both oxygen and the analyte of interest, often glucose, from the blood, bodily fluids, and/or tissue of the patient 12 to diffuse into and through the transduction matrix 226 retained within the reaction chamber 222. As so described, the oxygen and the analyte follow separate and unique (different) diffusion paths into and through the transduction matrix 226. Specific representative examples of such separate and unique (different) diffusion paths will be illustrated and described in greater detail herein below in relation to FIGS. 3A and 3B.

In some embodiments, the transduction matrix 226 can be comprised primarily of a sol-gel such as a hydrogel that can be embedded with one or more enzymes and/or catalysts that are configured to cause reaction with the oxygen and the analyte of interest. The oxygen and the analyte of interest are thus consumed or depleted, at least in part, through the enzymatic reactions that occur within the transduction matrix 226. In certain alternative embodiments, the transduction matrix 226 can include composites, wick-like materials, etc., instead of or in addition to the hydrogel. For example, in some alternative embodiments, the transduction matrix 226 can include a sol-gel that is non-polymer-based, such as a ceramic sol-gel, a glass-type sol-gel, a carbon sol-gel, an aerogel, or other sponge-like or porous materials, with the solvent or water removed using a process similar to freeze drying that removes the liquid without collapsing the gel. In still other alternative embodiments, the reaction chamber 222 can be stuffed with cotton or fiberglass, and the enzymes can be attached covalently to form the transduction matrix 226, thus foregoing any hydrogel.

The sensing polymer 224, such as an oxygen sensing polymer (OSP) in certain non-exclusive embodiments, can then utilize the energy from the energy source 228 in order to generate a signal that is representative of the partial pressure of oxygen remaining within the transduction matrix 226 and adjacent to the sensing polymer 224. The signal can then be sent back through the energy guide 218 to the sensor assembly 216 where the signal can be used by the sensor assembly 216 to determine the actual level (or volume) of the analyte of interest within the blood, bodily fluids, and/or tissue of the patient 12.

As described in greater detail herein below, the design of the sensor 214 enables the realization of many advantages and benefits in comparison to currently available CGM devices, such as (1) Minimization of Low Limit of Detection (LOD) and Non-Specific Adsorption, (2) Improvement in Reproducibility and Stability, and (3) Minimization of Biofouling, Fibrous Encapsulation, Inflammation, and Loss of Host Vasculature.

The energy guide 218 can have any suitable design for guiding the energy from the energy source 228 toward the remaining components of the sensor 214, depending on the specific requirements of the analyte sensing system 210, the sensor 214 and/or the sensor assembly 216.

As noted above, in many embodiments, the energy guide 218 can be provided in the form of an optical fiber that is configured to guide light energy from the energy source 228 of the sensor assembly 216 toward the remaining components of the sensor 214. The optical fiber can have a core and cladding layer that are made of any suitable materials, such as plastic and/or silica. In certain embodiments, the core of the optical fiber can encompass a hollow core photonic crystal fiber or a polymer light pipe, which can have a higher refractive index than a clad material that is found in a traditional optical fiber. This may also be a hollow glass or plastic capillary tube that is metalized internally. In some embodiments, the optical fiber can have certain buffer material stripped therefrom to get down to the size of a cladding layer. In certain non-exclusive embodiments, the cladding layer can have a size of approximately 125 micrometers (μm), although larger and smaller sizes can also be realized. It is appreciated, however, that with removal of the buffer, the optical fiber can be more susceptible to mechanical stress. Thus, in some embodiments, the sheath 220 can further function as a mechanical stress relief, in addition to providing the substantially tubular-shaped, oxygen permeable material that helps to define the reaction chamber 222 in which the transduction matrix 226 is retained.

In certain embodiments, the energy guide 218 (optical fiber) can further include a polymeric coating in the form of a thin, protective buffer layer 1172 (illustrated in FIG. 11A, and sometimes referred to as a “buffer layer” or a “buffer”) that is configured to enhance the structural integrity of the energy guide 218. More particularly, in some embodiments, the buffer layer 1172 may be retained on the energy guide 218 (such as the optical fiber) all the way to a guide distal end 218D where the sensing polymer 224 (OSP) can be coated. It is appreciated that, in such embodiments, the energy guide 218 would be inserted into the sheath 222 with the buffer layer 1172 remaining intact, without stripping.

As discussed in greater detail herein below, it is appreciated that the buffer layer 1172 can be formed at least partially from any suitable polymeric materials.

In some non-exclusive alternative embodiments, the energy guide 218 can be further provided with more than one protective buffer layer. For example, in one non-exclusive alternative embodiment, the energy guide 218 can include a first buffer layer (formed from a polymeric material such as polyimide), and then a second buffer layer (formed from a polymeric material such as acrylic, or other suitable polymeric material). In certain embodiments, the use of multiple buffer layers can be of greater benefit during the manufacturing process.

In some alternative embodiments, the energy guide 218 can be provided in a format other than as an optical fiber. For example, in certain non-exclusive alternative embodiments, if the energy source 228 is an electrical energy source, the energy guide 218 can include one or more electrodes that can be embedded into the sheath 220 for electrochemical sensing. Still alternatively, the energy guide 218 can have another suitable design.

In still other alternative embodiments, the sensor 214 can include more than one energy guide 218 for purposes of guiding the energy from the energy source 228 toward the sensing polymer 224 and/or the transduction matrix 226, and/or for subsequently transmitting returning energy or signal back toward the sensor assembly 216.

In yet other alternative embodiments, the sensor 214 can be provided without the particular requirement of the energy guide 218. In some embodiments, the sensor 214 can further include an energy transmission facilitator 1774 (illustrated in FIG. 17) of any suitable design that is configured to facilitate the transmission of the energy from the energy source 228 toward the sensing polymer 224 and/or the transduction matrix 226 without the particular requirement of the energy guide 218.

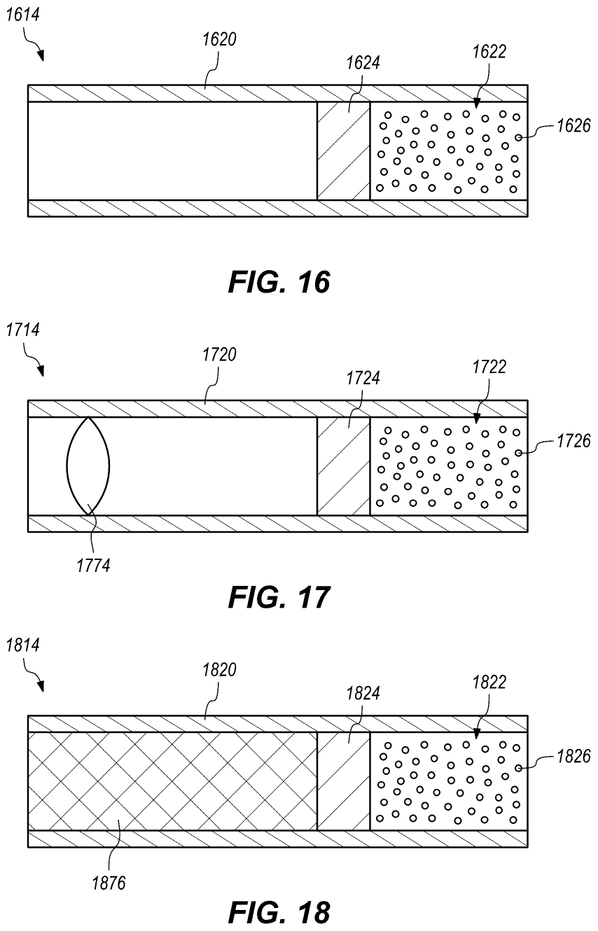

Certain examples of such alternative embodiments of the sensor 214, such as those designed without an energy guide or with more than one energy guide, are illustrated and described herein below in relation to FIGS. 16-19.

As illustrated, the sheath 220 is coupled to the energy guide 218 at or near a guide distal end 218D of the energy guide 218. As noted above, by coupling the sheath 220 at or near the guide distal end 218D of the energy guide 218, the sheath 220 can help to provide mechanical stress relief to the energy guide 218 so as to inhibit breaking of the energy guide 218.

In some embodiments, an adhesive 230 can be utilized to secure the sheath 220 to the energy guide 218 at or near the guide distal end 218D of the energy guide 218. The adhesive 230 can have any suitable design for purposes of helping to overcome unwanted diffusion events. In certain implementations, the adhesive 230 can be formed as an oxygen impermeable adhesive that further inhibits oxygen and the analyte of interest from entering into the reaction chamber 222 and/or into the transduction matrix 226 from a proximal end 220P of the sheath 220. Alternatively, the sheath 220 can be secured to the energy guide 218 by heating and melting the sheath 220 onto the energy guide 218. Still alternatively, a mechanical feature can be incorporated into the design to hold the sheath 220 in place relative to the energy guide 218.

The sheath 220 is configured to control the permeation of certain components of the blood, bodily fluids or tissue of the patient 12, such as oxygen and glucose (or other analyte of interest), into the reaction chamber 222 that is defined, at least in part, by the sheath 220. More specifically, in various embodiments, the sheath 220 can be a three-dimensional, substantially tubular-shaped (or cylindrical-shaped) sheath that is oxygen permeable, while also being impermeable to the analyte of interest. In certain embodiments, the sheath 220 can be configured to have at least approximately 180 degrees of oxygen permeability. In other embodiments, the sheath 220 can be configured to have a full 360 degrees of oxygen permeability. In still other embodiments, the sheath 220 can have a different degree of oxygen permeability.

The sheath 220 can be formed through any suitable manufacturing process. For example, in many embodiments, the sheath 220 can be formed through use of one of a three-dimensional extrusion technique and a three-dimensional molding technique. In such embodiments, the three-dimensional extruded or molded tubing of the sheath 220 can help make the sensor 214 as a whole more reliable, lower cost, less likely to fall apart, and less likely to prolapse after implantation. The sheath 220 can also encompass a concentric unibody design that can overcome limitations of a multi-layer design, as the need to glue layers together can increase sources of inaccuracy. The concentric unibody design can further eliminate complicated machining and assembly issues that could cause variation or lack of repeatability and reproducibility. Alternatively, the sheath 220 can be formed via another suitable manufacturing process.

It is appreciated that the size and shape of the sheath 220 are generally manufactured to provide a precise volume and/or geometry for the reaction chamber 222 and the transduction matrix 226 retained therein. As noted, the sheath 220 defines at least a portion of the reaction chamber 222. For example, as illustrated in this embodiment, the sheath 220 defines the annular sides as well as a chamber distal end 222D of the reaction chamber 222, while the sensing polymer 224 is positioned adjacent to the guide distal end 218D of the energy guide 218 in order to define a chamber proximal end 222P of the reaction chamber 222. In an alternative interpretation, the guide distal end 218D can be said to define the chamber proximal end 222P of the reaction chamber 222, and the sensing polymer 224 can be said to be positioned within the reaction chamber 222, adjacent to the guide distal end 218D and adjacent to the transduction matrix 226.

The precise volume of the reaction chamber 222 is thus defined by a chamber diameter 222A, which is equal to an inner diameter of the sheath 220, and a chamber length 222L, which extends from the chamber distal end 222D to the chamber proximal end 222P.

By adjusting the chamber diameter 222A and the chamber length 222L, the volume of the analyte of interest that enters into the reaction chamber 222 through the sheath distal end 2200 of the sheath 220 can be effectively controlled.

In some embodiments, the chamber diameter 222A can be between approximately 100 nanometers (nm) and 500 micrometers (μm). In other embodiments, the chamber diameter 222A can be between approximately one micrometer (μm) and 250 micrometers (μm). In still other embodiments, the chamber diameter 222A can be between approximately 15 micrometers (μm) and 150 micrometers (μm). In certain non-exclusive embodiments, the chamber diameter 222A can be approximately 100 nanometers (nm), 500 nanometers (nm), one micrometer (μm), five micrometers (μm), ten micrometers (μm), 25 micrometers (μm), 50 micrometers (μm), 125 micrometers (μm), 250 micrometers (μm), 320 micrometers (μm), or 500 micrometers (μm). Alternatively, the chamber diameter 222A can be greater than approximately 500 micrometers (μm) or less than approximately 100 nanometers (nm).

In certain embodiments, the chamber length 222L can be varied to adjust the overall range of sensitivity of the sensor 214. For example, in an environment with a relatively low level of analytes, if a sheath 220 with a longer chamber length 222L is used, then the analytes will typically be fully depleted as they diffuse through the transduction matrix 226 toward the sensing polymer 224 as a result of the enzyme reaction. It is noted that once the analyte of interest is depleted as a result of the enzyme reaction, there will be a zone behind this (toward the chamber proximal end 222P) where the level of oxygen in the reaction chamber 222 will begin to rise again due to the oxygen no longer being consumed through reaction with the analyte (with the analyte having been depleted before reaching this zone), and with the continual and constant diffusion of the oxygen through the sheath 220. Thus, in such an environment, it is generally preferred to use a sheath 220 with a shorter chamber length 222L in order to get a more accurate reading on the level of analytes.

Conversely, in an environment with a relatively high level of analytes, if a sheath 220 with a short chamber length 222L is used, then there would not be sufficient time or space for the enzyme reaction to deplete much of the analyte or the oxygen as they diffuse through the transduction matrix 226 toward the sensing polymer 224. Thus, in such an environment, it is generally preferred to use a sheath 220 with a longer chamber length 222L in order to get a more accurate reading on the level of analytes.

As so described, it is appreciated that the chamber length 222L and/or the positioning or depth of the sensing polymer 224 relative to the transduction matrix 226 is a critical tuning parameter for providing the sensor 214 with a desired sensitivity. Moreover, as described in greater detail herein below, based on such alternative environments within the patient, it is often preferred to utilize a multi-sensor channel, which can utilize sensors of different lengths within a single analyte sensing system 210. Such alternative designs enable the analyte sensing system 210 to provide a more accurate reading on the level of analytes regardless of whether the analyte sensing system 210 is being utilized in an environment with a relatively low level of analytes or a relatively high level of analytes.

It is further appreciated that the degree of oxygen permeability of the sheath 220 is defined by the specific material(s) that are used to form the sheath 220, including the density of such materials, as well as a wall thickness 220T of walls of the sheath 220. The chamber length 222L, the chamber diameter 222A, and the wall thickness 220T of the walls of the sheath 220 are important factors to regulate and control in order to improve the overall accuracy of the sensor 214. For example, the overall diffusion space available within the reaction chamber 222 can be controlled in a manner that effectively inhibits bubble formation, which could otherwise adversely impact the precision and accuracy of the sensor 214.

The sheath 220 can be formed from any suitable materials in order to effectively control radial diffusion of oxygen into the reaction chamber 222, and thus effectively tuning the sensitivity of the sensor 214. Moreover, the specific material of the sheath 220 can also help protect the guide distal end 218D (the fiber optic tip) of the energy guide 218 while implanted in the body 11 of the patient 12, and ensures that the energy guide 218 is removed from the body 11 when the sensor 214 is explanted, even if the guide distal end 218D (the fiber optic tip) breaks in-situ.

The material(s) used for the sheath 220 can be selected based on various factors, including biocompatibility, oxygen permeability, manufacturability, and lubriciousness. In certain non-exclusive embodiments, the sheath 220 can be formed from one or more of fluorinated ethylene propylene (FEP), paraformaldehyde (PFA), polytetrafluoroethylene (PTFE), ePTFE (such as Gore-Tex®), polyether block amide (PEBA), polyvinylchloride (PVC), polydimethylsiloxane, polyurethane, polyimide, polystyrene, sulfonated tetrafluoroethylene-based fluoropolymer-copolymer (such as Nafion®) and similar ionomers, and polycarbonate or blends or co-polymers or composites of these materials, woven or non-woven meshes such as Celgard® membrane or Tyvek®, and elastomers or reinforced elastomers such as polybutadiene polyisobutene, neoprene, isoprene, nitrile, styrene butadiene or co-polymers, blends or composites thereof. Alternatively, the sheath 220 can be formed from other suitable materials that also provide the desired control of oxygen permeability and diffusion, as well as biocompatibility to inhibit adverse reactions when the analyte sensing system 210 and/or the sensor 214 are used in-vivo. Such alternative materials include ceramics, sintered or porous metal tubing, carbon fiber, graphite or other materials with a porosity that is permeable to oxygen and impermeable to the analyte. Still alternatively, the sheath 220 can be formed from one or more distributed polyacetylenes bearing trimethylsilyl groups, such as poly(1-trimethylsilyl-1-propyne (PTMSP)), desilylated halogen containing diphenylacetylene co-polymers, copolymerization of FS and CS (1-(3,4-difluorophenyl)-2-(4-trimethylsilylphenyl) acetylene (FS) and 1-(3,4-dichlorophenyl)-2-(4-trimethylsilylphenyl) acetylene (CS)), diphenylacetylene copolymers, PFCS, and desilylated fluorocopolymer (DPFC)]

In certain implementations, lowering the oxygen permeability of the sheath 220 can make the sensor 214 more hyposensitive. More particularly, by thus reducing the amount of oxygen available in the transduction matrix 226, the oxygen will be consumed or depleted faster with smaller amounts of the analyte.

It is appreciated that the materials utilized for the sheath 220, which have a desired level of oxygen permeability, are also designed to be impermeable to the analyte of interest. More particularly, the sheath 220 is configured such that the analyte of interest cannot permeate and/or diffuse through the body of the sheath 220 and into the reaction chamber 222, and thus into the transduction matrix 226 retained therein. Rather, the only access for the analyte of interest into the reaction chamber 222, and thus into the transduction matrix 226, is through the sheath distal end 2200 of the sheath 220. With such design, the oxygen and the analyte of interest follow different and/or separate diffusion paths into and through the reaction chamber 222 and/or the transduction matrix 226 that is retained therein. More specifically, the oxygen will follow a diffusion path that is primarily radial relative to the sheath 220, the reaction chamber 222 and the transduction matrix 226, while the analyte will follow a diffusion path that is primarily longitudinal relative to the sheath 220, the reaction chamber 222 and the transduction matrix 226. However, as noted above, in some embodiments, the sheath 220 may be permeable to and/or conduct a small amount of the analyte of interest (such as glucose) to tune the gradient formed by the primary conduction path through the sheath distal end 220D, but not contributing a diffusion path greater than through the sheath distal end 220D. It is appreciated that in such alternative embodiments, the sheath 220 would still be considered to be “impermeable” to the analyte of interest.

It is appreciated that the sheath 220 can have any desired wall thickness 220T for purposes of controlling the oxygen permeability in a desired manner. For example, in certain non-exclusive embodiments, the sheath 220 can have a wall thickness 220T of between approximately ten micrometers (μm) and 400 micrometers (μm). In other embodiments, the sheath 220 can have a wall thickness 220T of between approximately one micrometer (μm) and 100 (μm). In still other embodiments, the sheath 220 can have a wall thickness 220T of between approximately ten micrometers (μm) and 75 micrometers (μm). Alternatively, the sheath 220 can have a wall thickness 220T that is greater than approximately 400 micrometers (μm) or less than approximately one micrometer (μm).

Although the materials of the sheath 220 must be taken into consideration, generally speaking, the greater the wall thickness 220T of the sheath 220, the lower the oxygen permeability will be through the sheath 220. It is appreciated that the wall thickness 220T of the walls of the sheath 220 can be easily controlled through the three-dimensional extrusion or molding technique that can be utilized to form the sheath 220 in many embodiments. Thus, the oxygen permeability through the sheath 220 can be easily controlled utilizing such manufacturing techniques. For example, forming the sheath 220 with thinner walls with such techniques serves to improve oxygen permeation, thus increasing gradient for a given hydrogel diffusion velocity.

As a whole, in various embodiments, it is desired that the sheath 220 exhibit a particular level of oxygen permeability in order to more effectively control the volume of materials and the reactions that occur within the reaction chamber 222. In some non-exclusive embodiments, the sheath 220 can be manufactured to provide an oxygen permeability of between approximately five (cm³ mm/m²·day·bar)/mm and 60,000 (cm³ mm/m²·day·bar)/mm. In other embodiments, the sheath 220 can provide an oxygen permeability of between approximately ten (cm³ mm/m²·day·bar)/mm and 10,000 (cm³ mm/m²·day·bar)/mm. In still other embodiments, the sheath 220 can provide an oxygen permeability of between approximately 20 (cm³ mm/m²·day·bar)/mm and 5,000 (cm³ mm/m²·day·bar)/mm. Alternatively, the sheath 220 can have a wall density that is greater than approximately 30,000 (cm³ mm/m²·day·bar)/mm, such as up to approximately 60,000 (cm³ mm/m²·day·bar)/mm in some embodiments, or less than approximately five (cm³ mm/m²·day·bar)/mm. It is appreciated, however, that smaller or lower oxygen permeability can have an adverse impact on sensor dynamic range. It is further appreciated that there may still be occasions where a smaller or lower oxygen permeability is preferable.

It is further appreciated that the design and manufacturing process for forming the sheath 220 can be effectively controlled in order to overcome precision and accuracy issues. For example, in some implementations, the sensor tip can be precisely cut with a razor blade, or similar sharp instrument, so that the sheath 220 and/or the reaction chamber 222 can be cut to precise lengths within +/−50 micrometers.

In some embodiments, the functional end of the sensor 214, including primarily the sheath 220, can be further covered with a coating 232 that is formed from a bio-compliant material or surface treatment that is designed to improve longevity when the analyte sensing system 210 and/or the sensor 214 are used in-vivo. In particular, the coating 232 can be effectively utilized to inhibit material separation and ensure biocompatibility protection. In one embodiment, the coating 232 can encompass a polyurethane-based treatment utilized on an outer surface 2200 of the sheath 220 to promote biocompatibility. In another embodiment, the coating 232 can be provided in the form of embossed structures that are formed onto the outer surface 2200 of the sheath 220 to ensure compatibility.

In the embodiment illustrated in FIG. 2A, the sensing polymer 224 is coated onto the guide distal end 218D of the energy guide 218. Alternatively, the sensing polymer 224 can be provided in another suitable format. For example, in one non-exclusive alternative embodiment, the sensing polymer 224 can be provided as a plurality of particulates, such as nanoparticles, that are dispersed throughout the transduction matrix 226.

The sensing polymer 224 is configured to receive energy from the energy source 228, and to use that energy to sense one or more components that are diffusing through the transduction matrix 226, at an interface between the sensing polymer 224 and the transduction matrix 226. More particularly, in various embodiments, the sensing polymer 224 is configured to generate a signal corresponding to the sensed components found within the blood, bodily fluids or tissue of the patient 12 as such components are diffusing through the transduction matrix 226 and arrive at or near the interface between the sensing polymer 224 and the transduction matrix 226.

In many embodiments, the sensing polymer 224 comprises an oxygen sensitive polymer (OSP) that is configured to sense oxygen within the transduction matrix 226, which is representative of a level of an analyte of interest within the blood, bodily fluids or tissue of the patient 12. In other embodiments, the sensing polymer 224 can be configured to sense pH levels within the transduction matrix 226, which, in such embodiments, are also representative of a level of an analyte of interest within the blood, bodily fluids or tissue of the patient 12. In still other embodiments, the sensing polymer 224 can be configured to directly sense the level of the analyte of interest from the blood, bodily fluids or tissue of the patient 12 that is diffusing through the transduction matrix 226.

In certain embodiments, the purpose of the OSP is to evaluate and/or analyze the transduction matrix 226 and to generate a signal in the form of a phosphorescent, triplet state whose amplitude and lifetime are proportional to a lack of oxygen. Oxygen partial pressure will quench the excited state causing the lifetime (or amplitude) to be shorter dependent on the partial pressure concentration of oxygen. The OSP is also sensitive to temperature, but the spectrum incorporated within the signal can give an indication of the temperature independent of the oxygen quenching. In certain embodiments, lifetime is chosen as the basis of the measurement as opposed to amplitude, as amplitude can be noisier and prone to losses in the signal path. However, it is appreciated that either lifetime or amplitude could work for purposes of evaluating a lack (or depletion) of oxygen within the transduction matrix 226.

As described, the OSP works based on the principle of phosphorescence triplet quenching of a “fluorescent” dye molecule that is covalently bound or entrapped in an oxygen permeable but diffusion limiting or regulating transduction matrix 226. Alternatively, detection can also be conducted by luminescent means, including bioluminescence by firefly or bacterial luciferase enzymes, or chemiluminescence with horseradish peroxidase.

As described in greater detail herein below, the transduction matrix 226 can be a polymer or co-polymer that controls the diffusion rate, and thus the quenching rate constant of the dye. Molecular oxygen immediately brings the excited state of a phosphorescent dye back to ground state, causing emission of a characteristic spectrum of light, specific to the particular dye used. Without the presence of oxygen, the dye has a natural lifetime, characteristic of the dye molecule(s) chosen. The dye excited by a short pulse of light will either decay after a time, naturally or this decay time will be shortened by collisional (non-radiative} transfer of the energy stored in the triplet state of the dye, or “quenched”. The higher the partial pressure of the quencher, the shorter the decay time of the excited dye.

From the perspective of the dye emission signal, either intensity (amplitude) or lifetime (pulse width of the phosphorescence decay, or phase shift of the excitation light to the return signal fluorescence or phosphorescence) can be used for purposes of evaluating or sensing the consumption or depletion of oxygen within the transduction matrix 226. In certain implementations, intensity can be prone to fluctuations in amplitude which could occur from bending losses in the energy guide 218 (optical fiber) or similar loss mechanisms. Thus, lifetime can be a more preferred method to give a reliable signal devoid of amplitude fluctuations or noise. It is appreciated that the relationship of oxygen to lifetime is not linearly proportional, but rather is a curve, due to effects such as multiple microdomains, averaging, self-quenching of the dye, dimer formation, and other mechanisms that lead to non-linearity. Alternatively, a combination of these two signals and/or a spectroscopic ratio between wavelengths (colors) of the phosphorescent signal could also be used. Alternatively, a fluorescent or other (such as colorimetric) on/off indicator may be used if there are enough channels distributed in the sheath 220 to give a reasonable resolution from multiple channels, such that the sensor 214 is digital in nature as opposed to having an analog lifetime or amplitude measurement associated with each channel that may overlap in its lifetime or amplitude sensing range. A digital on/off sensor would not overlap and require relatively close spacing, but still be distant enough from its adjacent channels to give a gradient. This would be analogous to sampling. The indicator may be reversible, as in the case of a fluorescent quenching indicator, or may be latching and a permanent change. This could also be cumulative in the case of a colorimetric that once a threshold of detection is reached or a comparator reference level achieved, a “true” or “on” state for that channel is indicated.

It is appreciated that it can be difficult to directly measure glucose (or other analyte of interest) using spectroscopy. Therefore, in many embodiments, the sensor 214 can be configured to measure the glucose (or other analyte of interest) indirectly by evaluating the consumption or depletion of background oxygen in the transduction matrix 226 based on a two substrate (oxygen+analyte) enzymatic reaction. This creates a two-step process to measure glucose (or other analyte) in interstitial fluid and may necessitate subtracting any variation in oxygen partial pressure (concentration or dissolved oxygen in interstitial fluid) by way of a reference channel that measures oxygen only. The reference channel provides a differential signal to “baseline out” the oxygen reading variability of the tissue site being measured.

In some embodiments, the sensing polymer 224 can be made hydrophobic to reduce signal quenching by water by using polymers that are naturally hydrophobic. It is appreciated that most polymers (even hydrophobic ones) tend to absorb 1-2% water. The hydrophobic design of the sensing polymer 224 can help to overcome signal degradation from hydration issues related to the transduction matrix 226, which is typically a hydrophilic component. Alternatively, or in addition, additives can further be included for purposes of overcoming free radical and antioxidants, singlet delta oxygen quenchers or signal degradation including, but not limited to, Betahydroxytoluene (BHT), Betahydroxyacetone (BHA), quercetin, and 1,4-Diazabicyclo[2.2.2]octane (DABCO) as non-exclusive examples.

In certain embodiments, adhesives can be utilized to promote adhesion of the sensing polymer 224, such as the OSP, to the energy guide 218. Alternatively, the energy guide 218 can be treated, such as with a silane treatment, in order to promote adhesion of the sensing polymer 224 to the energy guide 218. Still alternatively, adhesion between the sensing polymer 224 and the energy guide 218 can be accomplished in another suitable manner such as etching by hydrofluoric acid, fluoride compounds, sodium or potassium hydroxide. or laser microstructuring the energy guide, plasma etching, or by sandblasting.

The transduction matrix 226 is positioned within the reaction chamber 222, as defined at least in part by the sheath 220, and is configured to provide a location for the different or separate paths of diffusion for the oxygen and the analyte of interest that have permeated through the body of the sheath 220 (i.e. the path of the oxygen) or through the sheath distal end 220D of the sheath 220 (i.e. a path for both oxygen and the analyte of interest). For any embodiment, the transduction matrix 226 will have a volume that is defined by the chamber length 222L and the chamber diameter 222A of the reaction chamber 222. Generally speaking, with a smaller chamber diameter 222A, less analyte is let into the reaction chamber 222 and there is a corresponding increase in the oxygen-to-analyte ratio. Conversely, with a larger chamber diameter 222A, more analyte is let into the reaction chamber 222 and there is a corresponding decrease in the oxygen-to-analyte ratio.

As noted above, the chamber length 222L of the reaction chamber 222, and the precise positioning of the sensing polymer 224 therein, as well as the different or separate paths of diffusion for the oxygen and the analyte of interest through the transduction matrix 226 help to enhance the overall accuracy and reliability of the sensor 214.

The transduction matrix 226 functions as a “wick” using capillary action and diffusion to conduct interstitial fluid through the sensor 214 and is a support for an enzyme and/or catalyst. As the analyte (glucose, lactate, ketones, cholesterol, pyruvates, alcohol, bilirubin, xanthenes, citrates, etc.) diffuses through the transduction matrix 226, it is progressively consumed. In the case of two or more substrate enzymatic reactions, such as glucose and molecular oxygen, both substrates diffuse through the transduction matrix 226. Thus, as described, the transduction matrix 226 is where the analyte-specific chemical reactions occur, which consume or deplete the oxygen and the analyte. Moreover, the material makeup of the transduction matrix 226, as well as the permeability limiting/controlling design of the sheath 220, provides for a rate or flux limitation of oxygen that is different than that of the analyte of interest thereby creating a detectable gradient due to the speed of conduction of the oxygen versus the analyte, and the reaction rate of the catalyst.

The transduction matrix 226 can be formed from any suitable materials. In many embodiments, the transduction matrix 226 can be comprised of a hydrogel that is embedded with enzymes (or other reactive chemistry), catalysts, co-factors, and/or that consumes oxygen or produces a luminescent reaction. Additionally, detection can also be accomplished via luminescent means including bioluminescence by firefly or bacterial luciferase enzymes, or chemiluminescence with horseradish peroxidase.

Alternatively, in certain embodiments, the transduction matrix 226 can be hydrophilic in order to overcome analyte diffusion limitations. The transduction matrix 226 can further include a filler or wick within the hydrogel to promote stability in the capillary forces, as well as overcoming undesired hydrogel swelling and to promote rehydration of dried hydrogel. In many embodiments the gel macromers or monomers may consist of xanthan gum, polyacrylic acid, acrylamide, PAGE gels used in electrophoresis (N,N-dimethyl acrylamide/acrylamide), agarose, gelatin, polyvinylpyrrolidone, polyvinyl alcohol (PVA), alginate, and silicone hydrogels similar to formulations used in soft contact lenses, etc.

The repeatability of the defining of the chamber diameter 222A through the specific manufacturing process utilized to form the sheath 220 helps to define a consistent hydrogel cross-sectional area for the transduction matrix 226 and an oxygen consumption cone/gradient. This is a critical component that contributes to the consistent amount of transduction matrix 226 between sensors, which in turn reduces any sensor to sensor variation. In other embodiments, the cross-sectional size and shape of the reaction chamber 222 can change along the chamber length 222L. For example, as opposed to the consistent cross-sectional size and shape of the reaction chamber 222 shown in FIG. 2A, in certain non-exclusive alternative embodiments, the reaction chamber 222 can be tapered toward the chamber distal end 222D, flared out toward the chamber distal end 222D to shape the gradient and to linearize the analyte detection gradient. In still other embodiments, the reaction chamber 222 can be cut at an angle (non-perpendicular) at the chamber distal end 222D. In similar fashion, the wall thickness or density of the sheath material can be tapered from thick to thin, or thin to thick, or any other variation or combination of thicknesses, to change the gas diffusion rate though the sidewall to shape or linearize the gradient.

As described, many embodiments presume an enzymatic reaction. The hydrogel may be selective in its diffusion to preferentially allow the analyte to diffuse through the hydrogel while blocking interfering agents that might provide a false positive response. More particularly, similar to the hydrophobic nature of the sensing polymer 224, the hydrogel can be configured to block water, an interfering quencher, along with any dissolved ions in said water.

In embodiments of the present invention where the analyte sensing system 210 is used for a glucose sensing application, the enzyme included within the transduction matrix 226 is glucose oxidase (GOx). In alternative embodiments Glucose Hexokinase may be used. The hexokinase enzyme in the presence of Adenosine triphosphate (ATP) converts glucose to glucose-6-phosphate. Glucose-6-phosphate and the cofactor nicotinamide adenine dinucleotide phosphate, oxidized form (NADP+), are converted to 6-phosphogluconate and nicotinamide adenine dinucleotide phosphate, reduced form (NADPH), by glucose-6-phosphate dehydrogenase. NADPH has a strong absorbance maximum at 340 nm. The appearance rate of NADPH can be monitored spectrophotometrically and is proportional to the amount of glucose in the sample. Other methods for detection of glucose examples would be the Glucose dehydrogenase method.

The glucose oxidase (or GOx) sensor material catalytically reacts glucose and oxygen, consuming the reactants and producing a product (such as gluconolactone, gluconic acid, hydrogen peroxide, water, etc.) upon contact with glucose and oxygen. In some embodiments, the sensor material may also include a synthetic or inorganic (non-enzymatic) catalyst such as gold nanoparticles (or other suitable nanozymes). Thus, the GOx substantially simultaneously consumes or reacts with the glucose and the oxygen. More specifically, GOx works by catalytically reacting glucose and molecular oxygen to produce glucono-δ-lactone and hydrogen peroxide. The hydrogen peroxide would degrade the sensor enzyme and the sensing polymer dye, so an enzyme (catalase) is added to break down the hydrogen peroxide, immediately, into oxygen and water to prevent the reactive peroxide from degrading the enzyme. The products of this reaction and any side products produced, diffuse out of the sensor 214 into the body. These products are naturally produced in the body and are metabolized further, and eliminated. Alternatively, or in addition, other materials that are free radical scavengers and/or antioxidants can be added to prolong the life of the enzyme such as BHT, BHA, Quercetin, and 1,4-Diazabicyclo[2.2.2]octane (DABCO) as non-exclusive examples.

As so described, the GOx serves as a reaction-generating substrate at the lead point of the sensor 214 that functions as a catalyst upon contact with oxygen and the analyte of interest. Such reactions can be generated in three dimensions, with the first two dimensions including the oxygen diffusing through the sidewalls of the sheath 220, but the final dimension including the oxygen directly entering the sheath 220 through the sheath distal end 220D in front of the GOx, rather than indirectly entering through the body of the sheath 220. As further noted above, in certain embodiments, the sheath 220 may leak some amount of the analyte of interest through the sheath 220 to tune the diffusion gradient, while still being considered to be “impermeable” to the analyte of interest.

Referring briefly to FIGS. 3A and 3B, FIGS. 3A and 3B illustrate theoretical analyte and oxygen profiles within the reaction chamber 222 (illustrated in FIG. 2A), as defined at least in part by the sheath 220, and/or within the transduction matrix 226 (illustrated in FIG. 2A). More specifically, FIG. 3A is a representative illustration showing a theoretical analyte profile 340A that may be realized as the analyte follows its diffusion path during use of the analyte sensing system 210 illustrated in FIG. 2A; and FIG. 3B is a representative illustration showing a theoretical oxygen profile 340B that may be realized as the oxygen follows its diffusion path during use of the analyte sensing system 210 illustrated in FIG. 2A.

It is appreciated that as the overall level of analyte concentration increases, and diffuses through the transduction matrix 226, the less oxygen will still exist (non-reacted and non-depleted) at the interface with the sensing polymer 224. As a result, there will be a corresponding change in the signal that is sent from the sensor 214 (illustrated in FIG. 2A) and/or the sensing polymer 224 back through the energy guide 218 to the sensor assembly 216 (illustrated in FIG. 2A). In particular, when there is a lot of glucose (or other analyte of interest), the analyte can travel essentially the full chamber length 222L (illustrated in FIG. 2A) of the reaction chamber 222 and consume almost all of the available oxygen via the enzyme reaction. Therefore, the sensing polymer 224 will sense a minimal amount of oxygen. Conversely, when there is very little glucose (or other analyte of interest), the analyte is quickly consumed and will not travel deep into the reaction chamber 222 due to the enzyme reaction. Therefore, the sensing polymer 224 will sense a much higher amount of available oxygen.

Returning back to FIG. 2A, in certain embodiments, the transduction matrix 226 can include additional constituents in order to inhibit other issues from adversely impacting the proper functioning of the sensor 214 and/or to promote certain desired attributes for the sensor 214. For example, in some embodiments, the transduction matrix 226 can incorporate one or more of (1) fillers such as hydroxyapatite, cellulose or fiberglass to help with drying/support matrix as well as to act as a wick for rehydration; (2) hydrating agents (glycerin, calcium chloride, deliquiscents/humectants, etc.), alternatively the hydrogel may be dried, preserving the gel structure by way of freeze drying or critical point drying to prevent the gel from collapsing; (3) adhesion promoters such as 3-aminopropyl methacrylamide hydrochloride, APTES, 3-(trimethoxysilyl)propyl methacrylate or other suitable silane coupling agents for acrylic sensing polymers or an appropriate adhesion promoter for alternate sensing polymer such as styrene, vinyl, etc. (for sensing polymer-to-fiber bonding); and (4) sterilization materials such as chlorhexidine sterilant and/or e-beam sterilization. It is appreciated that the use of e-beams may further crosslink the hydrogel in addition to sterilizing the sensor, and could be used to tune the diffusion velocity of the hydrogel by increasing crosslinking sites. E-beam crosslinking, UV crosslinking and other forms of crosslinking via grayscale lithography or multiphoton polymerization/initiation may also be used to create a gradient along the length of the transduction matrix 226 allowing for creation of a diffusion gradient that is non-linear or logarithmic. This could further be used to correct for the natural non-linear diffusion gradient in the transduction matrix 226 that has a homogeneous degree of crosslinking. This can also be used to tune sensitivity for different zones.

In some embodiments, the packaging may further include humectants, humidifiers and/or a liquid sterilant, such as chlorhexidine gluconate solution and/or phosphate buffered saline, to keep the hydrogel fully hydrated.

In certain embodiments, the transduction matrix 226 can be designed to incorporate an optimal cross linker to overcome analyte diffusion limitations and improve precision and accuracy of the sensor 214. The degree of crosslinking of the transduction matrix 226, along with the chamber diameter 222A, tune the diffusion velocity of the analyte, thereby shaping the oxygen, in conjunction with the diffusion path length. In certain non-exclusive embodiments, citrates, glutaraldehyde or formaldehyde, polyaziridines, freeze-thaw, e-beam or radiation crosslinking epichlorohydrin, amine active multifunctional crosslinkers, carboiimide, ionic crosslinkers such as iron nitrate, calcium chloride, and/or citric acid can be included to crosslink the enzyme to the hydrogel or to crosslink the gel macromers to entrap the enzyme within a gel. Ideally, a crosslinker would be used that covalently bonds the enzyme to the hydrogel as opposed to physical entrapment alone, thereby preventing leaching out of the catalyst or enzyme or nanozyme material. One examplar method can be functionalizing the enzyme with 2-Iminothiolane (Traut's reagent) to add sulfhydryl (S—H) functional groups to the enzyme that would be crosslinked by polyaziridine to the hydrogel macromer, for example. The degree of functionalization of the primary amine groups (such as amino acids) of common enzymes can be determined, for example, by the Ellman's Reagent protocol commonly known by those skilled in the art. Any leaching out of the catalyst or enzyme could present a biocompatibility issue by leaking these materials into the body or bloodstream potentially, as well as the issue of changing the Michaelis Menten curve of the enzyme or catalyst reaction rate of the hydrogel/catalyst combination. Mass flow controllers can also be used to tune the sensors more effectively and accurately dial in the proper cross linker range(s).

As noted herein, the sheath 220 can have an open sheath distal end 220D to enable the analyte to effectively enter into and diffuse through the transduction matrix 226. However, it is appreciated that it is desired to maintain the transduction matrix 226 within the sheath 220 and/or within the reaction chamber 222. Keeping the transduction matrix 226 within the sheath 220 and/or within the reaction chamber 222 can be enabled in a number of different manners. For example, in one embodiment, the sheath 220 can further include an optional end cap, plug or film covering, that keeps the transduction matrix 226 within the reaction chamber 222 while still allowing the analyte to enter into the reaction chamber 222. In another embodiment, an etching process can be used to improve surface tension on the inside of the sheath 220 so that the transduction matrix 226 is less likely to ooze out. In still other embodiments, the crosslinking of the transduction matrix 226 can be included so that the transduction matrix 226 is firm enough to stay inside the reaction chamber 222. In such embodiments, the transduction matrix 226 will be more solid, while being balanced enough to allow the analyte to diffuse fast enough for a true gradient and be reasonably usable for in tissue measurement. In yet other embodiments, the chamber diameter 222D can be small enough so as to add the benefit of surface tension to help enable the transduction matrix 226 to stay within the reaction chamber 222. In still yet other embodiments, the manufacturing of the sheath 220 can be such as to inhibit the transduction matrix 226 from getting air bubbles and/or drying out, and thereby further enable the transduction matrix 226 to stay within the reaction chamber 222. In some embodiments a vent or port at the more proximal end near the OSP, to help facilitate rehydration and prevent trapping of bubbles. This port would need to have a membrane that selectively allows liquid and/or gas permeation, without passing any glucose or analyte molecules. Such a membrane may be glued over the port or may be deposited in a liquid form and cured or dried.

During in vivo use of the analyte sensing system 210, the sensor assembly 216 is positioned adjacent to the epidermis 11A (illustrated in FIG. 1) of the patient 12, and the sensor 214 extends into the body 11 (illustrated in FIG. 1) of the patient 12 and beneath the epidermis 11A of the patient 12. Once positioned in a desired manner, the energy source 228 generates and/or emits energy that is directed through the energy guide 218 toward the sensing polymer 224. The sensing polymer 224 absorbs the energy from the energy source 228, and then emits or generates a signal, such as a phosphorescence signal in certain embodiments, that is directed back through the energy guide 218 toward the sensor assembly 216. The emitted or generated signal will vary depending on the local oxygen concentration level sensed by the sensing polymer 224, which is directly proportional to the amount of oxygen consumed through the transduction matrix 226. Stated in another manner, the emitted or generated signal is based on the reaction-generating materials of the transduction matrix 226, which consumes or depletes both the oxygen and the analyte of interest diffusing along different or separate paths through the transduction matrix 226. The sensed level of oxygen remaining at the interface with the sensing polymer 224 can then be used by the sensor assembly 216, through an algorithm embedded therein, to determine the level of the analyte of interest that is present within the blood, bodily fluids or tissue of the patient 12.

FIG. 2B is a sectional view illustration of the analyte sensing system 210 taken on line 2B-2B in FIG. 2A. In particular, FIG. 2B is a sectional view illustration of the analyte sensing system 210 that shows the energy guide 218, the sheath 220, and the coating 232 that can be formed about the sheath 220.

FIG. 2C is a sectional view illustration of the analyte sensing system 210 taken on line 2C-2C in FIG. 2A. In particular, FIG. 2C is a sectional view illustration of the sheath 220, which defines the reaction chamber 222 therein that is filled with the transduction matrix 226, and the coating 232 that can be formed about the sheath 220.

As described, in certain embodiments, the sensor 214 is a single channel sensor that is configured to sense an analyte of interest within the blood, bodily fluids or tissue of the patient 12. As such, the sensor 214 can sometimes be referred to as a sensor channel. However, in various embodiments of the present invention, the sensor 214 can be made with multiple channels in multiple configurations. For example, in certain non-exclusive alternative embodiments, the present invention can incorporate one or more of: (1) Multiple energy guides that can be bundled with multiple tubes, a larger single tube, or a multi-lumen tube; (2) Separate tubes or lumens that can have different reagents for the transduction matrix to detect different analytes; (3) Tubes or lumens that can extend to different lengths from the sensing polymer to change the diffusion length for the analyte and thus tune the channel to a specific sensitivity range; and (4) One or more energy guides that can be used as a reference channel to measure the baseline oxygen level in the environment.

In various alternative embodiments, the analyte sensing system can include different tubing configurations, such as (i) single lumen, (ii) separate lumens being used for each channel with the same or different material to tune the sensitivity for each channel or to measure a separate analyte in each channel, (iii) a large single lumen can be used to bundle more than one fiber optic element, (iv) multi-lumen tubes can be used to keep the fibers bundle, and/or (v) each lumen can have one or more fiber optic elements.

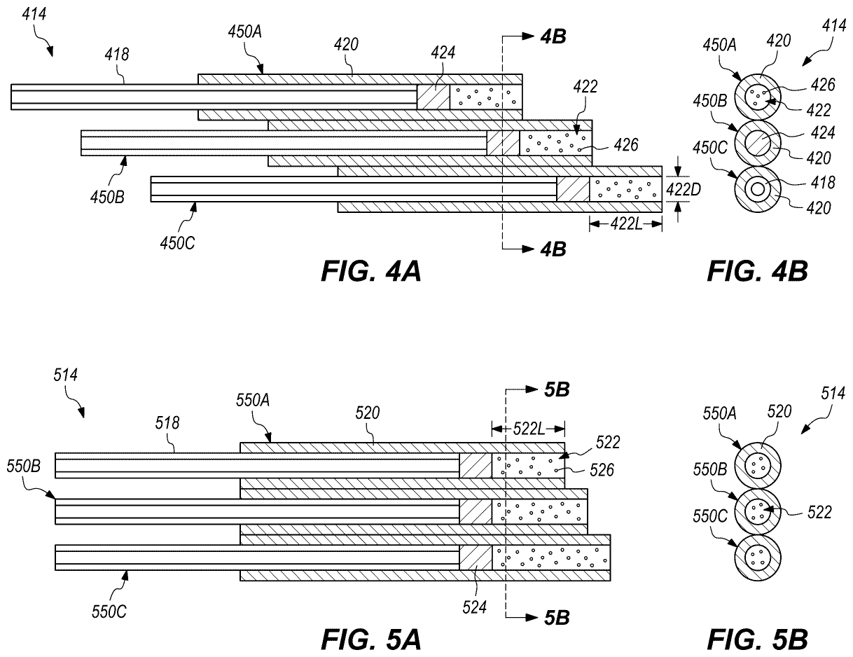

Thus, in many embodiments, the analyte sensing system 210 can be configured to incorporate a multi-channel, optical-based sensor, with each sensor channel having a design that is similar to the design of the sensor 214 described in detail in relation to FIG. 2A. Multi-channel sensors, such as described herein, can thus generate multiple analyte (such as glucose or other suitable analyte) paths versus a singular analyte path for traditional, generally available single-channel sensors. Certain non-exclusive alternative embodiments of such multi-channel, optical-based sensors are illustrated and described herein below in relation to FIGS. 4A-9C. As noted above, it is appreciated that while the present invention is often directed toward optical-based sensors, where the energy guide will typically be provided in the form of an optical fiber (with the energy source being a light source), the energy guide can alternatively be provided in another format, such as one or more electrodes embedded in the tubes for electrochemical sensing. It is further appreciated that, while FIGS. 4A-9C describe certain specific alternative embodiments of the multi-channel, optical-based sensor, features of any of the individual embodiments can be combined in any suitable manner to create even more embodiments of the sensor, provided such modifications are still in accordance with the overall teachings set forth in the present specification.

FIG. 4A is a simplified schematic illustration of another embodiment of the sensor 414 that can be included as part of the analyte sensing system 210 (illustrated in FIG. 2A). In particular, in this embodiment, the sensor 414 is a multi-channel, multi-fiber and multi-tube sensor, with staggered tip locations.

As illustrated in FIG. 4A, the sensor 414 includes a first sensor channel 450A, a second sensor channel 450B, and a third sensor channel 450C, which can be positioned substantially adjacent to one another in any suitable manner. As further illustrated, each sensor channel 450A-450C has an overall design that is substantially similar to the sensor 214 illustrated and described in relation to FIG. 2A. More specifically, as shown, each sensor channel 450A-450C includes an energy guide 418, a sheath 420 that defines at least a part of a reaction chamber 422, a sensing polymer 424, and a transduction matrix 426 that are substantially similar to the energy guide 218, the sheath 220, the sensing polymer 224, and the transduction matrix 226 illustrated and described herein above. Accordingly, a description of such components will not be repeated in detail in relation to FIG. 4A.

It is further noted that the reaction chamber 422 for each of the individual sensor channels 450A-450C has a similar, if not identical, chamber length 422L and chamber diameter 422D. Thus, the transduction matrix 426 retained therein will have a volume that is similar, if not identical, for each of the sensor channels 450A-450C.

However, FIG. 4A also shows that the chamber distal end 422D for each of the reaction chambers 422 are staggered relative to one another, such that the reaction chambers 422 will be positioned at slightly different depths within the body 11 (illustrated in FIG. 1) of the patient 12 (illustrated in FIG. 1) when being used in vivo. As such, each of the sensor channels 450A-450C will record individual levels of analytes at different locations within the body 11 of the patient 12. Such design enables improved accuracy and reliability as each sensor channel 450A-450C can be used as a check on each of the other sensor channels 450A-450C, and potential outliers can be removed from the analysis if it is determined that they are likely inaccurate.

It is further appreciated that each sensor channel 450A-450C can have the same or different material within the transduction matrix 426 to tune the sensitivity for each sensor channel 450A-450C and/or to measure a separate analyte in each sensor channel 450A-450C.

FIG. 4B is a sectional view illustration of the sensor 414 taken on line 4B-4B in FIG. 4A. As shown, the first sensor channel 450A on line 4B-4B cuts through the sheath 420 and the reaction chamber 422, with the transduction matrix 426 retained therein. The second sensor channel 450B on line 4B-4B cuts through the sheath 420 and the sensing polymer 424. The third sensor channel 450C on line 4B-4B cuts through the sheath 420 and the energy guide 418.

FIG. 5A is a simplified schematic illustration of still another embodiment of the sensor 514 that can be included as part of the analyte sensing system 210 (illustrated in FIG. 2A). In particular, in this embodiment, the sensor 514 is a multi-channel, multi-fiber and multi-tube sensor, with various lengths for the reaction chamber 522 within the individual tubes.

As illustrated in FIG. 5A, the sensor 514 includes a first sensor channel 550A, a second sensor channel 550B, and a third sensor channel 550C, which can again be positioned substantially adjacent to one another in any suitable manner. As further illustrated, each sensor channel 550A-550C has an overall design that is substantially similar to the sensor 214 illustrated and described in relation to FIG. 2A. More specifically, as shown, each sensor channel 550A-550C includes an energy guide 518, a sheath 520 that defines at least a part of the reaction chamber 522, a sensing polymer 524, and a transduction matrix 526 that are substantially similar to the energy guide 218, the sheath 220, the sensing polymer 224, and the transduction matrix 226 illustrated and described herein above. Accordingly, a description of such components will not be repeated in detail in relation to FIG. 5A.

However, as noted above, each of the sensor channels 550A-550C has a chamber length 522L of the reaction chamber 522 that is different than for each of the other sensor channels 550A-550C. With the reaction chamber 522 for each sensor channel 550A-550C thus extending to different lengths from the sensing polymer 524, the diffusion length for the analyte within the reaction chamber 522 is also changed, and, thus, the sensor channel 550A-550C can be tuned to a specific sensitivity range.

As with the previous embodiment, it is further appreciated that each sensor channel 550A-550C can have the same or different material within the transduction matrix 526 to tune the sensitivity for each sensor channel 550A-550C and/or to measure a separate analyte in each sensor channel 550A-550C.

FIG. 5B is a sectional view illustration of the sensor 514 taken on line 5B-5B in FIG. 5A. As shown, line 5B-5B cuts through the sheath 520 and the reaction chamber 522 in each of the first sensor channel 550A, the second sensor channel 550B, and the third sensor channel 550C.

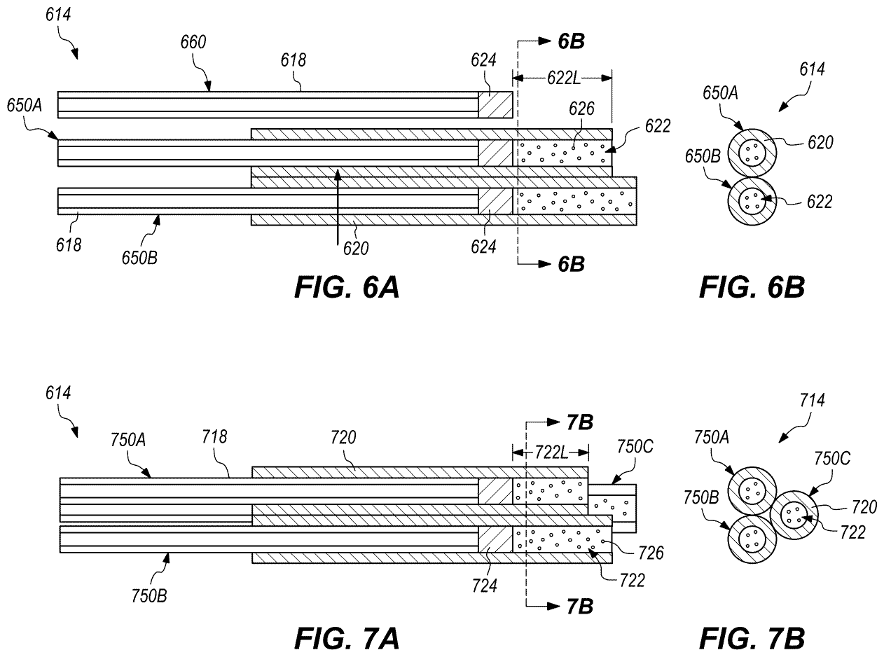

FIG. 6A is a simplified schematic illustration of another embodiment of the sensor 614 that can be included as part of the analyte sensing system 210 (illustrated in FIG. 2A). In particular, in this embodiment, the sensor 614 is a multi-channel, multi-fiber and multi-tube sensor, which also includes a reference channel 660 that directly senses the level of oxygen without the oxygen being diffused through the sheath 620 and into and through the transduction matrix 626 and without the oxygen reacting with enzymes and/or the analyte of interest.

As illustrated in FIG. 6A, the sensor 614 includes a first sensor channel 650A, and a second sensor channel 650B, with each sensor channel 650A-650B having an overall design that is substantially similar to the sensor 214 illustrated and described in relation to FIG. 2A. More specifically, as shown, each sensor channel 650A-650B includes an energy guide 618, a sheath 620 that defines at least a part of a reaction chamber 622, a sensing polymer 624, and a transduction matrix 626 that are substantially similar to the energy guide 218, the sheath 220, the sensing polymer 224, and the transduction matrix 226 illustrated and described herein above. Accordingly, a description of such components will not be repeated in detail in relation to FIG. 6A.

It is noted that each of the sensor channels 650A-650B again has a different chamber length 622L of the reaction chamber 622, such that each of the sensor channels 650A-650B can be tuned to a specific sensitivity range. As with the previous embodiments, it is further appreciated that each sensor channel 650A-650B can have the same or different material within the transduction matrix 626 to tune the sensitivity for each sensor channel 650A-650B and/or to measure a separate analyte in each sensor channel 650A-650B.

However, as noted above, in this embodiment, the sensor 614 further includes the reference channel 660 which simply includes the energy guide 618 and the sensing polymer 624. With such design, the sensing polymer 624 is configured to directly sense the level of oxygen within the blood, bodily fluids, and/or tissue of the patient 12 (illustrated in FIG. 1), without the oxygen being diffused through a sheath and/or into and through a transduction matrix, and without the oxygen reacting with enzymes and/or the analyte of interest. This effectively sets a baseline level of oxygen that can be utilized to more accurately determine how much oxygen has been consumed or depleted in the transduction matrix 626 in the sensor channels 650A-650B. Moreover, the reference channel 660 can further monitor oxygen and/or temperature to verify site health, namely oxygen availability, which is an indication of site health. The sensor 614 can then send error messages when the readings should not be relied on and/or indicate when the sensor 614 may no longer have any analyte-diffusing capable tissue. In some alternative embodiments, the sensor 614 can include more than one reference channel 660, or the reference channel 660 can be split into multiple reference channels.

FIG. 6B is a sectional view illustration of the sensor 614 taken on line 6B-6B in FIG. 6A. As shown, line 6B-6B cuts through the sheath 620 and the reaction chamber 622 in each of the first sensor channel 650A, and the second sensor channel 650B. Line 6B-6B further cuts directly adjacent to the sensing polymer 624 (illustrated in FIG. 6A) for the reference channel 660 (illustrated in FIG. 6A), such that no structure is shown for the reference channel 660 in FIG. 6B.

FIG. 7A is a simplified schematic illustration of yet another embodiment of the sensor 714 that can be included as part of the analyte sensing system 210 (illustrated in FIG. 2A). In particular, in this embodiment, the sensor 714 again is a multi-channel, multi-fiber and multi-tube sensor, with various lengths for the reaction chamber 722 within the individual tubes, similar to the embodiment in FIG. 5A, but where the tubes are bundled together in a 3-D arrangement (rather than being merely positioned in a side-by-side manner).

More specifically, as illustrated in FIG. 7A, the sensor 714 again includes a first sensor channel 750A, a second sensor channel 750B, and a third sensor channel 750C, with each sensor channel 750A-750C again having an overall design that is substantially similar to the sensor 214 illustrated and described in relation to FIG. 2A. More specifically, as shown, each sensor channel 750A-750C again includes an energy guide 718, a sheath 720 that defines at least a part of the reaction chamber 722, a sensing polymer 724, and a transduction matrix 726 that are substantially similar to the energy guide 218, the sheath 220, the sensing polymer 224, and the transduction matrix 226 illustrated and described herein above. Each of the sensor channels 750A-750C again has a chamber length 722L of the reaction chamber 722 that is different than for each of the other sensor channels 750A-750C such that the sensor channel 750A-750C can be tuned to a specific sensitivity range. Again, as with the previous embodiments, it is further appreciated that each sensor channel 750A-750C can have the same or different material within the transduction matrix 726 to tune the sensitivity for each sensor channel 750A-750C and/or to measure a separate analyte in each sensor channel 750A-750C.

However, as noted, in this embodiment, the sensor channels 750A-750C are arranged in a different manner relative to one another than in the embodiment shown in FIG. 5A. More particularly, the sensor channels 750A-750C are bundled together in a three-dimensional arrangement, rather than being merely positioned next to one another in a side-by-side arrangement.

FIG. 7B is a sectional view illustration of the sensor 714 taken on line 7B-7B in FIG. 7A. As shown, line 7B-7B cuts through the sheath 720 and the reaction chamber 722 in each of the first sensor channel 750A, the second sensor channel 750B, and the third sensor channel 750C. FIG. 7B also more clearly illustrates the three-dimensional arrangement of the sensor channels 750A-750C relative to one another.

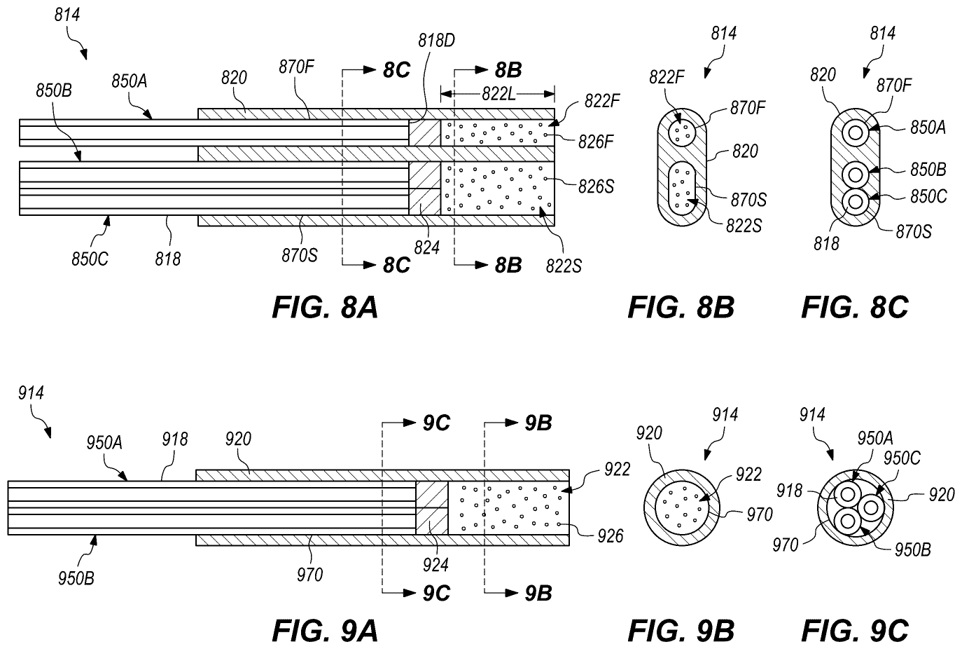

FIG. 8A is a simplified schematic illustration of another embodiment of the sensor 814 that can be included as part of the analyte sensing system 210 (illustrated in FIG. 2A). In particular, in this embodiment, the sensor 814 is a multi-channel, multi-fiber sensor, with only a single tube, but where the tube includes multiple lumens, with one or more fibers extending into each of the lumens.

More specifically, as illustrated in FIG. 8A, the sensor 814 includes a first sensor channel 850A, a second sensor channel 850B, and a third sensor channel 850C, with each sensor channel 850A-850C including an energy guide 818 and a sensing polymer 824 that is coated and/or secured onto the guide distal end 818D of the energy guide 818. The energy guide 818 and the sensing polymer 824 for each of the sensor channels 850A-850C are substantially similar to what has been illustrated and described in detail herein above.

However, in this embodiment, all three of the sensor channels 850A-850C are provided within a single sheath 820, with the first sensor channel 850A being provided and/or functioning within a first sheath lumen 870F, and the second sensor channel 850B and the third sensor channel 850C being provided and/or functioning within a second sheath lumen 870S. As further shown in FIG. 8A, the first sheath lumen 870F of the sheath 820 defines at least a part of a first reaction chamber 822F that retains a first transduction matrix 826F therein. Somewhat similarly, the second sheath lumen 870S of the sheath 820 defines at least a part of a second reaction chamber 822S that retains a second transduction matrix 826S therein. It is appreciated that the second sensor channel 850B and the third sensor channel 850C can provide redundancy for one another in determining a level of the analyte of interest as they both extend into or through the same second sheath lumen 870S and utilize the same second reaction chamber 822S and the same second transduction matrix 826S.

It is appreciated that, as illustrated, each of the first reaction chamber 822F and the second reaction chamber 822S can have a similar chamber length 822L. Alternatively, however, the first reaction chamber 822F and the second reaction chamber 822S can have different chamber lengths from one another, such as by adjusting the positioning of the energy guide 818 and the sensing polymer 824 relative to the sheath 820 and/or relative to the particular sheath lumens 870F, 870S into or through which they extend.

Similar to previous embodiments, it is further appreciated that each reaction chamber 822F, 822S can have the same or different material within the corresponding transduction matrix 826F, 826S to tune the sensitivity for the sensor channels 850A-850C and/or to measure a separate analyte in each reaction chamber 822F, 822S.

FIG. 8B is a sectional view illustration of the sensor 814 taken on line 8B-8B in FIG. 8A. As shown, line 8B-8B cuts through the sheath 820 to illustrate the first sheath lumen 870F that defines at least a part of the first reaction chamber 822F, and the second sheath lumen 870S that defines at least a part of the second reaction chamber 822S.

FIG. 8C is a sectional view illustration of the sensor 814 taken on line 8C-8C in FIG. 8A. As shown, line 8C-8C cuts through the sheath 820 to illustrate the first sheath lumen 870F, with the energy guide 818 of the first sensor channel 850A extending therethrough, and the second sheath lumen 870S, with the energy guides 818 of the second sensor channel 850B and the third sensor channel 850C extending therethrough.

FIG. 9A is a simplified schematic illustration of still yet another embodiment of the sensor 914 that can be included as part of the analyte sensing system 210 (illustrated in FIG. 2A). In particular, in this embodiment, the sensor 914 is a multi-channel, multi-fiber sensor with the fibers being bundled together in a 3-D arrangement, somewhat similar to the embodiment shown in FIG. 7A, but with only a single tube such that each of the fibers extending into the single tube.

More specifically, in the embodiment shown in FIG. 9A, the sensor 914 includes a first sensor channel 950A, a second sensor channel 950B, and a third sensor channel 950C (not shown in FIG. 9A, but illustrated in FIG. 9C), with each sensor channel 950A-950C including an energy guide 918 and a sensing polymer 924 that is coated and/or secured onto the guide distal end 918D of the energy guide 918. The energy guide 918 and the sensing polymer 924 for each of the sensor channels 950A-950C are substantially similar to what has been illustrated and described in detail herein above.

However, in this embodiment, all three of the sensor channels 950A-950C are provided within a single sheath 920 that defines a single sheath lumen 970. With such design, each of the first sensor channel 950A, the second sensor channel 950B and the third sensor channel 950C are provided and/or function within the sheath lumen 970. As further shown in FIG. 9A, the sheath lumen 970 of the sheath 920 defines at least a part of a reaction chamber 922 that retains a transduction matrix 926 therein. It is appreciated that the sensor channels 950A-950C can provide redundancy in determining a level of the analyte of interest as they each extend into or through the same sheath lumen 970 and utilize the same reaction chamber 922 and the same transduction matrix 926.

FIG. 9B is a sectional view illustration of the sensor 914 taken on line 9B-9B in FIG. 9A. As shown, line 9B-9B cuts through the sheath 920 to illustrate the sheath lumen 970 that defines at least a part of the reaction chamber 922.

FIG. 9C is a sectional view illustration of the sensor 914 taken on line 9C-9C in FIG. 9A. As shown, line 9C-9C cuts through the sheath 920 to illustrate the sheath lumen 970, with the energy guide 918 of each of the sensor channels 950A-950C extending therethrough.

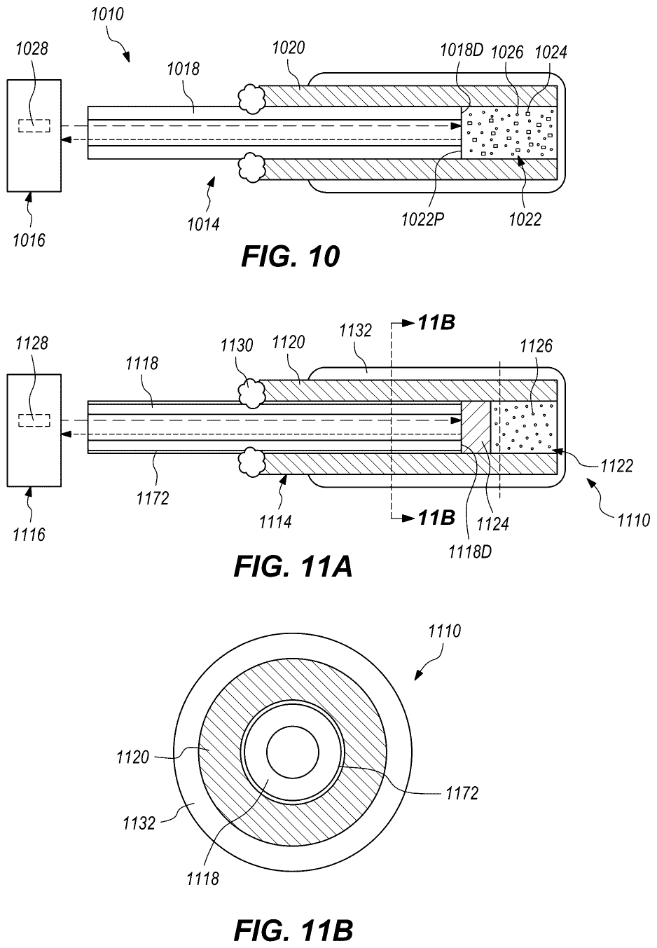

As noted above, the sensing polymer can be provided in a different configuration than has been described in detail herein above. In particular, FIG. 10 is a simplified schematic illustration of another embodiment of the analyte sensing system 1010, including still another embodiment of the sensor 1014, where the sensing polymer 1024 is provided in an alternative format.

The analyte sensing system 1010 is substantially similar in design and function to the embodiments of the analyte sensing system 210 illustrated and described herein above. In particular, the analyte sensing system 1010 again includes a sensor 1014 that is configured to sense an analyte, such as glucose or another suitable analyte, within the blood, bodily fluids, and/or tissue of the patient 12 (illustrated in FIG. 1), and a sensor assembly 1016 (illustrated as a box) that receives a signal from the sensor 1014 regarding the sensed analyte to determine a level (or volume) of the analyte within the blood, bodily fluids, and/or tissue of the patient 12. The sensor 1014 again includes one or more of (i) an energy guide 1018, (ii) a sheath 1020 that defines at least a portion of a reaction chamber 1022, (iii) a sensing polymer 1024, and (iv) a transduction matrix 1026 that can be received and retained substantially, if not entirely, within the reaction chamber 1022; and the sensor assembly 1016 can again include an energy source 1028 (illustrated as a box in phantom) that is configured to generate energy that is guided by and/or directed through the energy guide 1018 of the sensor 1014.

However, as noted, in this embodiment, the sensing polymer 1024 is being provided in a different format than in the previous embodiments. More specifically, instead of the sensing polymer 1024 being provided as a coating that is secured adjacent to the guide distal end 1018D of the energy guide 1018 as has been illustrated and described in many embodiments herein above, the sensing polymer 1024 is provided in micro or nano particulate form that is distributed throughout and/or within the transduction matrix 1026. For example, in one non-exclusive embodiment, the sensing polymer 1024 can be provided in the form of nanoparticles that are distributed throughout and/or within the transduction matrix 1026. Thus, in this embodiment, the guide distal end 1018D of the energy guide 1018 would define the chamber proximal end 1022P of the reaction chamber 1022, and the sensing polymer 224 would be positioned within the reaction chamber 1022.

With such design, the sensing polymer 1024 is still configured to receive energy from the energy source 228 that has been guided through the energy guide 1018, and to use that energy to sense one or more components that are diffusing through the transduction matrix 1026 at an interface between the particulates of the sensing polymer 1024 and the transduction matrix 1026. The sensing polymer 1024 will again generate a signal corresponding to the sensed components found within the blood, bodily fluids, and/or tissue of the patient 12 as such components are diffusing through the transduction matrix 1026 and arrive at or near the interface between the particulates of the sensing polymer 1024 and the transduction matrix 1026.

FIG. 11A is a simplified schematic cutaway view illustration of still another embodiment of the analyte sensing system 1110 illustrated in FIG. 1, including still another embodiment of the sensor 1114. As illustrated, the analyte sensing system 1110 and the sensor 1114 are substantially similar to the analyte sensing system 210 and the sensor 214 illustrated and described herein above in relation to FIG. 2A. In particular, the analyte sensing system 1110 again includes the sensor 1114 that includes one or more of (i) an energy guide 1118, (ii) a sheath 1120 that defines at least a portion of a reaction chamber 1122, (iii) a sensing polymer 1124, and (iv) a transduction matrix 1126 that can be received and retained substantially, if not entirely, within the reaction chamber 1122; and a sensor assembly 1116 (illustrated as a box) that includes an energy source 1128 (illustrated as a box in phantom) that is configured to generate energy that is guided by and/or directed through the energy guide 1118 of the sensor 1114 toward the sensing polymer 1124 and/or the transduction matrix 1126. Additionally, in some embodiments, an adhesive 1130 can again be utilized to secure the sheath 1120 to the energy guide 1118 at or near the guide distal end 1118D of the energy guide 1118. Further, in certain embodiments, the functional end of the sensor 1114, including primarily the sheath 1120, can again be further covered with a coating 1132 that is formed from a bio-compliant material or surface treatment that is designed to improve longevity when the analyte sensing system 1110 and/or the sensor 1114 are used in-vivo. Such noted components are substantially similar to what has been illustrated and described in detail herein above. Accordingly, a detailed description of such components will not be repeated in relation to FIG. 11A.

However, as shown in this embodiment, the sensor 1114 further includes a protective buffer layer 1172 (sometimes referred to as a “buffer layer” or a “buffer”) that is positioned about the energy guide 1118 in order to enhance the structural integrity of the energy guide 1118. In particular, the buffer layer 1172 helps to provide a certain level of protection and toughness for the energy guide 1118 as the sensor 1114 is being moved into the body of the patient for use during a diagnostic procedure.

As shown in this embodiment, the buffer layer 1172 may be retained on the energy guide 1118 (such as an optical fiber) all the way to the guide distal end 1118D where the sensing polymer 1124 (such as OSP) can be coated. It is appreciated that, in this embodiment, the energy guide 1118 would be inserted into the sheath 1122 with the buffer layer 1172 remaining intact, without stripping.

The buffer layer 1172 can be formed from any suitable materials. For example, in certain non-exclusive alternative embodiments, the buffer layer 1172 can be formed at least partially from one or more polymeric materials, such as acrylic, polyimide, polyimide acrylate, fluoroacrylate, silicones, carbon, polyether ether ketone (PEEK), polybutylene terephthalate (PBT), polyethylene terephthalate (PET), polyethylene, polyvinyl chloride (PVC), polyvinylidene fluoride (PVDF), polyurethane, TPU polyurethanes, copolyester elastomer (such as Hytrel®), ethylene tetrafluoroethylene (ETFE), polytetrafluoroethylene (PTFE, such as Teflon®), perfluoroalkoxy alkane (PFA), or polypropylene. Alternatively, the buffer layer 1172 can be formed from other suitable materials.

In many embodiments, the buffer layer 1172 can be retained on the energy guide 1118 over the cladding to help inhibit the energy guide 1118 (optical fiber) from breaking. It is appreciated that it is generally preferred to have a thin buffer layer 1172 (polymer coating) so as to keep the sensor opening from being too large. In some embodiments, the buffer layer 1172 can further include a dye incorporated therein in order to block optical interferents, as well as to improve noise levels and increase sensor accuracy.

FIG. 11B is a sectional view illustration of the analyte sensing system 1110 taken on line 11B-11B in FIG. 11A. In particular, FIG. 11B is a sectional view illustration of the analyte sensing system 1110 that shows the energy guide 1118, the buffer layer 1172 that can be positioned about the energy guide 1118, the sheath 1120, and the coating 1132 that can be formed about the sheath 1120.

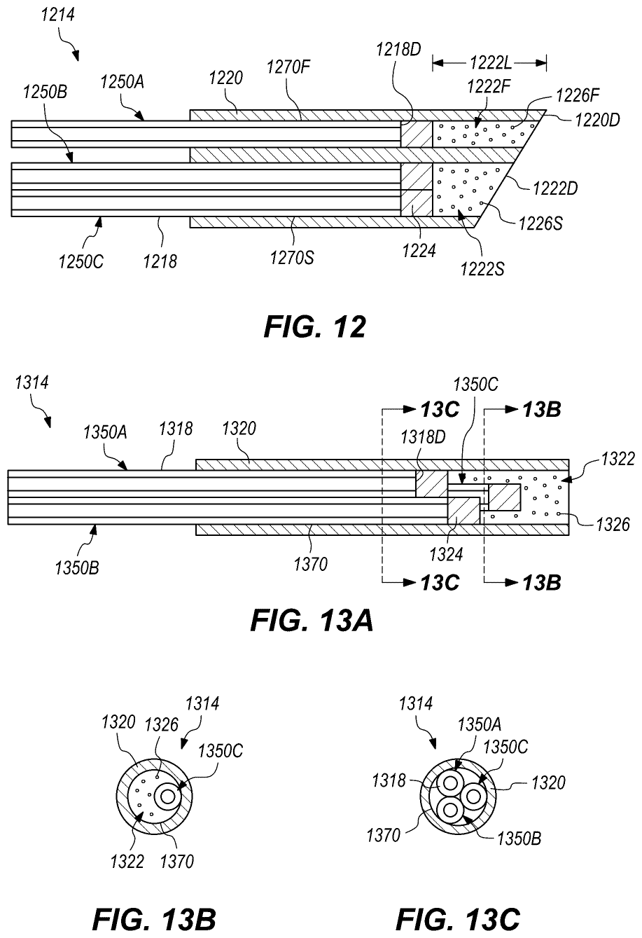

FIG. 12 is a simplified schematic illustration of yet another embodiment of the sensor 1214 that can be included as part of the analyte sensing system 210 (illustrated in FIG. 2A). As illustrated, the sensor 1214 is substantially similar to the sensor 814 that was illustrated and described in detail above in relation to FIG. 8A.

In particular, in this embodiment, the sensor 1214 again is a multi-channel, multi-fiber sensor, with only a single tube, but where the tube includes multiple lumens, with one or more fibers extending into each of the lumens. More specifically, as illustrated in FIG. 12, the sensor 1214 again includes a first sensor channel 1250A, a second sensor channel 1250B, and a third sensor channel 1250C, with each sensor channel 1250A-1250C again including an energy guide 1218 and a sensing polymer 1224 that is coated and/or secured onto the guide distal end 1218D of the energy guide 1218. The energy guide 1218 and the sensing polymer 1224 for each of the sensor channels 1250A-1250C are substantially similar to what has been illustrated and described in detail herein above. Moreover, in this embodiment, all three of the sensor channels 1250A-1250C are again provided within a single sheath 1220, with the first sensor channel 1250A again being provided and/or functioning within a first sheath lumen 1270F, and the second sensor channel 1250B and the third sensor channel 1250C again being provided and/or functioning within a second sheath lumen 1270S.

As further shown in FIG. 12, the first sheath lumen 1270F of the sheath 1220 again defines at least a part of a first reaction chamber 1222F that retains a first transduction matrix 1226F therein. Somewhat similarly, the second sheath lumen 1270S of the sheath 1220 again defines at least a part of a second reaction chamber 1222S that retains a second transduction matrix 1226S therein. It is appreciated that the second sensor channel 1250B and the third sensor channel 1250C can again provide redundancy for one another in determining a level of the analyte of interest as they both extend into or through the same second sheath lumen 1270S and utilize the same second reaction chamber 1222S and the same second transduction matrix 1226S.

Similar to previous embodiments, it is further appreciated that each reaction chamber 1222F, 1222S can have the same or different material within the corresponding transduction matrix 1226F, 1226S to tune the sensitivity for the sensor channels 1250A-1250C and/or to measure a separate analyte in each reaction chamber 1222F, 1222S.

However, in this embodiment, the distal end 1220D of the sheath 1220 has been cut at an angle (non-perpendicular) such that the chamber distal end 1222D of each of the reaction chambers 1222F, 1222S also extends at an angle (is non-perpendicular) relative to the chamber length 1222L. With such design, it is appreciated that the first reaction chamber 1222F and the second reaction chamber 1222S can have different chamber lengths from one another, and the chamber length 1222L also varies from one side of the respective lumen 1270F, 1270S to the other side. It is further appreciated that by cutting the distal end 1220D of the sheath 1220 at such an angle, the volume of the analyte and/or the volume of oxygen entering through the chamber distal end 1222D of each of the reaction chambers 1222F, 1222S can be impacted (likely increased). This is because, with such design, the overall surface area of the chamber distal end 1222D is somewhat greater than the embodiment illustrated in FIG. 8A in which the distal end 1220D of the sheath 1220 is cut perpendicularly relative to the chamber length 1222L.

FIG. 13A is a simplified schematic illustration of still another embodiment of the sensor 1314 that can be included as part of the analyte sensing system 210 (illustrated in FIG. 2A). In particular, in this embodiment, the sensor 1314 is a multi-channel, multi-fiber sensor with the fibers being bundled together in a 3-D arrangement, somewhat similar to the embodiment shown in FIG. 7A, but with only a single tube such that each of the fibers extends into the single tube. More specifically, in the embodiment shown in FIG. 13A, the sensor 1314 again includes a first sensor channel 1350A, a second sensor channel 1350B, and a third sensor channel 1350C, with each sensor channel 1350A-1350C including an energy guide 1318 and a sensing polymer 1324 that is coated and/or secured onto the guide distal end 1318D of the energy guide 1318. The energy guide 1318 and the sensing polymer 1324 for each of the sensor channels 1350A-1350C are substantially similar to what has been illustrated and described in detail herein above.

However, in this embodiment, all three of the sensor channels 1350A-1350C are provided within a single sheath 1320 that defines a single sheath lumen 1370. With such design, each of the first sensor channel 1350A, the second sensor channel 1350B and the third sensor channel 1350C are provided and/or function within the sheath lumen 1370. As further shown in FIG. 13A, the sheath lumen 1370 of the sheath 1320 defines at least a part of a reaction chamber 1322 that retains a transduction matrix 1326 therein. As further shown in FIG. 13A, each of the sensor channels 1350A-1350C are positioned to extend to a different longitudinal position within the reaction chamber 1322. With such design, it is appreciated that the sensor channels 1350A-1350C are tuned to a specific sensitivity range, with the diffusion length for the analyte within the reaction chamber 1322 being somewhat different for each of the sensor channels 1350A-1350C.

FIG. 13B is a sectional view illustration of the sensor 1314 taken on line 13B-13B in FIG. 13A. As shown, line 13B-13B cuts through the sheath 1320 to illustrate the sheath lumen 1370 that defines at least a part of the reaction chamber 1322, and the transduction matrix 1326 that is retained substantially within the reaction chamber 1322. As further shown in FIG. 13B, at the longitudinal position where line 13B-13B cuts through the sheath 1320, line 13B-13B further cuts through the third sensor channel 1350C.

FIG. 13C is a sectional view illustration of the sensor 1314 taken on line 13C-13C in FIG. 13A. As shown, line 13C-13C cuts through the sheath 1320 to illustrate the sheath lumen 1370, with the energy guide 1318 of each of the sensor channels 1350A-1350C extending therethrough.

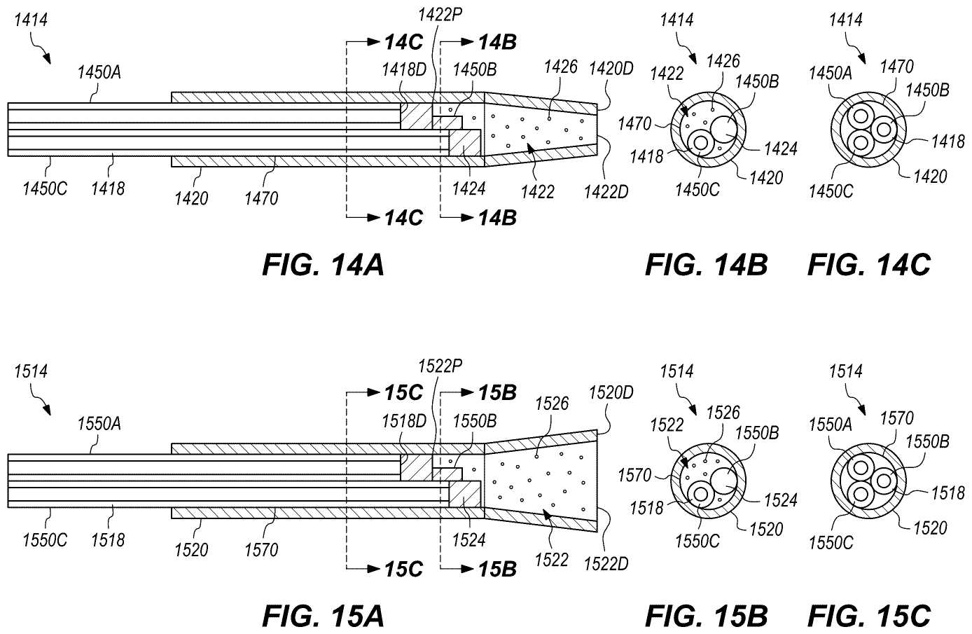

FIG. 14A is a simplified schematic illustration of still another embodiment of the sensor 1414 that can be included as part of the analyte sensing system 210 (illustrated in FIG. 2A). As illustrated, the sensor 1414 is substantially similar to the sensor 1314 illustrated and described in relation to FIG. 13A. In particular, in this embodiment, the sensor 1414 is again a multi-channel, multi-fiber sensor with the fibers being bundled together in a 3-D arrangement, somewhat similar to the embodiment shown in FIG. 7A, and again with only a single tube such that each of the fibers extending into the single tube. More specifically, in the embodiment shown in FIG. 14A, the sensor 1414 again includes a first sensor channel 1450A, a second sensor channel 1450B, and a third sensor channel 1450C, with each sensor channel 1450A-1450C including an energy guide 1418 and a sensing polymer 1424 that is coated and/or secured onto the guide distal end 1418D of the energy guide 1418. The energy guide 1418 and the sensing polymer 1424 for each of the sensor channels 1450A-1450C are substantially similar to what has been illustrated and described in detail herein above.

Additionally, in this embodiment, all three of the sensor channels 1450A-1450C are again provided within a single sheath 1420 that defines a single sheath lumen 1470. With such design, each of the first sensor channel 1450A, the second sensor channel 1450B and the third sensor channel 1450C are again provided and/or function within the single sheath lumen 1470. As further shown in FIG. 14A, the sheath lumen 1470 of the sheath 1420 again defines at least a part of a reaction chamber 1422 that retains a transduction matrix 1426 therein. As further shown in FIG. 14A, each of the sensor channels 1450A-1450C are again positioned to extend to a different longitudinal position within the reaction chamber 1422. With such design, it is appreciated that the sensor channels 1450A-1450C again can be tuned to a specific sensitivity range, with the diffusion length for the analyte within the reaction chamber 1422 being somewhat different for each of the sensor channels 1450A-1450C.

However, in this embodiment, the distal end 1420D of the sheath 1420 has been modified so as to impact the amount of the analyte of interest and the oxygen that can enter into the reaction chamber 1422 through the chamber distal end 1422D. More specifically, as shown in FIG. 14A, the distal end 1420D of the sheath 1420 has been tapered so that a lower amount of the analyte of interest and the oxygen can enter into the reaction chamber 1422 through the chamber distal end 1422D as compared to previous embodiments where the reaction chamber has a consistent diameter from the chamber proximal end to the chamber distal end. Stated in another manner, in this embodiment, less of the analyte of interest and the oxygen can enter into the reaction chamber 1422 through the chamber distal end 1422D than if the reaction chamber 1422 maintained the same diameter from the chamber proximal end 1422P to the chamber distal end 1422D.

FIG. 14B is a sectional view illustration of the sensor 1414 taken on line 14B-14B in FIG. 14A. As shown, line 14B-14B cuts through the sheath 1420 to illustrate the sheath lumen 1470 that defines at least a part of the reaction chamber 1422, and the transduction matrix 1426 that is retained substantially within the reaction chamber 1422. As further shown in FIG. 14B, at the longitudinal position where line 14B-14B cuts through the sheath 1420, line 14B-14B further cuts through the sensing polymer 1424 of the second sensor channel 1450B, and the energy guide 1418 of the third sensor channel 1350C.

FIG. 14C is a sectional view illustration of the sensor 1414 taken on line 14C-14C in FIG. 14A. As shown, line 14C-14C cuts through the sheath 1420 to illustrate the sheath lumen 1470, with the energy guide 1418 of each of the sensor channels 1450A-1450C extending therethrough.

FIG. 15A is a simplified schematic illustration of still another embodiment of the sensor 1514 that can be included as part of the analyte sensing system 210 (illustrated in FIG. 2A). As illustrated, the sensor 1514 is again substantially similar to the sensor 1314 illustrated and described in relation to FIG. 13A. In particular, in this embodiment, the sensor 1514 is again a multi-channel, multi-fiber sensor with the fibers being bundled together in a 3-D arrangement, somewhat similar to the embodiment shown in FIG. 7A, and again with only a single tube such that each of the fibers extending into the single tube. More specifically, in the embodiment shown in FIG. 15A, the sensor 1514 again includes a first sensor channel 1550A, a second sensor channel 1550B, and a third sensor channel 1550C, with each sensor channel 1550A-1550C including an energy guide 1518 and a sensing polymer 1524 that is coated and/or secured onto the guide distal end 1518D of the energy guide 1518. The energy guide 1518 and the sensing polymer 1524 for each of the sensor channels 1550A-1550C are substantially similar to what has been illustrated and described in detail herein above.

Additionally, in this embodiment, all three of the sensor channels 1550A-1550C are again provided within a single sheath 1520 that defines a single sheath lumen 1570. With such design, each of the first sensor channel 1550A, the second sensor channel 1550B and the third sensor channel 1550C are again provided and/or function within the single sheath lumen 1570. As further shown in FIG. 15A, the sheath lumen 1570 of the sheath 1520 again defines at least a part of a reaction chamber 1522 that retains a transduction matrix 1526 therein. As further shown in FIG. 15A, each of the sensor channels 1550A-1550C are again positioned to extend to a different longitudinal position within the reaction chamber 1522. With such design, it is appreciated that the sensor channels 1550A-1550C again can be tuned to a specific sensitivity range, with the diffusion length for the analyte within the reaction chamber 1522 being somewhat different for each of the sensor channels 1550A-1550C.

However, in this embodiment, the distal end 1520D of the sheath 1520 has been modified in a different manner so as to impact the amount of the of interest and the oxygen that can enter into the reaction chamber 1522 through the chamber distal end 1522D. More specifically, as shown in FIG. 15A, the distal end 1520D of the sheath 1520 has been flared so that a higher amount of the analyte of interest and the oxygen can enter into the reaction chamber 1522 through the chamber distal end 1522D as compared to previous embodiments where the reaction chamber has a consistent diameter from the chamber proximal end to the chamber distal end. Stated in another manner, in this embodiment, more of the analyte of interest and the oxygen can enter into the reaction chamber 1522 through the chamber distal end 1522D than if the reaction chamber 1522 maintained the same diameter from the chamber proximal end 1522P to the chamber distal end 1522D.

FIG. 15B is a sectional view illustration of the sensor 1514 taken on line 15B-15B in FIG. 15A. As shown, line 15B-15B cuts through the sheath 1520 to illustrate the sheath lumen 1570 that defines at least a part of the reaction chamber 1522, and the transduction matrix 1526 that is retained substantially within the reaction chamber 1522. As further shown in FIG. 15B, at the longitudinal position where line 15B-15B cuts through the sheath 1520, line 15B-15B further cuts through the sensing polymer 1524 of the second sensor channel 1550B, and the energy guide 1518 of the third sensor channel 1550C.

FIG. 15C is a sectional view illustration of the sensor 1514 taken on line 15C-15C in FIG. 15A. As shown, line 15C-15C cuts through the sheath 1520 to illustrate the sheath lumen 1570, with the energy guide 1518 of each of the sensor channels 1550A-1550C extending therethrough.

As noted above, certain embodiments of the sensor can be configured without the particular need for an energy guide. Stated in another manner, in some embodiments, any potential use of an energy guide can be as a separate and/or independent component from the sensor itself, with the use and functionality of the energy guide being solved via system integration rather than by specifically incorporating the energy guide into the sensor itself.

For example, FIG. 16 is a simplified schematic illustration of another embodiment of the sensor 1614 that can be included as part of the analyte sensing system 210 (illustrated in FIG. 2A), with the sensor 1614 being configured without an energy guide specifically incorporated therein. In certain embodiments, an energy guide can be subsequently provided as a separate element from the sensor 1614 itself, with its positioning and functionality being solved via system integration, rather than by being specifically incorporated into the initial design of the sensor 1614.

In particular, as illustrated in FIG. 16, the sensor 1614 again includes a sheath 1620 that defines at least a part of a reaction chamber 1622 that retains a transduction matrix 1626 therein, and a sensing polymer 1624 that is positioned within the sheath 1620. In this embodiment, the sensing polymer 1624 can be provided simply as a piece of sensing material that is inserted into the sheath 1620.

In contrast to previous embodiments, in the embodiment illustrated in FIG. 16, there is no energy guide that is provided as part of the sensor 1614 itself to guide the energy from the energy source 228 (illustrated in FIG. 2A) toward the sensing polymer 1624 and/or the transduction matrix 1626. Rather, the energy from the energy source 228 is simply directed into the sheath 1620 and thus toward the sensing polymer 1624 and/or the transduction matrix 1626. In certain such embodiments, an energy guide can be provided as a separate element from the sensor 1614 itself, in order to function in a manner similar to the embodiments illustrated and described in detail herein above in which the energy guide is specifically included as part of the sensor.

FIG. 17 is a simplified schematic illustration of still another embodiment of the sensor 1714 that can be included as part of the analyte sensing system 210 (illustrated in FIG. 2A), with the sensor 1714 again being configured without the specific inclusion of an energy guide. In particular, as illustrated in FIG. 17, the sensor 1714 again includes a sheath 1720 that defines at least a part of a reaction chamber 1722 that retains a transduction matrix 1726 therein, and a sensing polymer 1724 that is positioned within the sheath 1720. In this embodiment, the sensing polymer 1724 can again be provided simply as a piece of sensing material that is inserted into the sheath 1720.

However, in this embodiment, in order to facilitate the directing of the energy from the energy source 228 (illustrated in FIG. 2A) toward the sensing polymer 1724 and/or the transduction matrix 1726 without the sensor 1714 further including an energy guide, the sensor 1714 further includes an energy transmission facilitator 1774 (also referred to herein as a “transmission facilitator”) to facilitate such directing of the energy from the energy source 228.

The design of the transmission facilitator 1774 can be varied to suit the requirements of the sensor 1714. In one embodiment, the transmission facilitator 1774 can be provided in the form of a short segment of fiber, or a light pipe that can help direct the energy from the energy source 228 toward the sensing polymer 1724 and/or the transduction matrix 1726. In another embodiment, the transmission facilitator 1774 can be provided in the form of a piece of glass or plastic that can act as a window to the sensing polymer 1724 and/or the transduction matrix 1726. In still another embodiment, the transmission facilitator 1774 can be provided in the form of a gradient index (GRIN) lens. In yet another embodiment, the transmission facilitator 1774 can be provided in the form of a ball lens. In other embodiments, the transmission facilitator 1774 can be provided in the form of a plano-convex, concave-convex, or other focusing lens. In any of these noted embodiments, it is appreciated that the transmission facilitator 1774 helps to provide a relatively clear optical path to the sensing polymer 1724 and/or the transduction matrix 1726.

In still yet other embodiments, the transmission facilitator 1774 can be provided in the form of a double convex lens fiber optic that has had its cladding removed or stripped. In this embodiment, the core can further be formed into a hairpin or small radius loop. In some embodiments, the sensing polymer 1724 can be coated onto the cladding or core if the cladding has been removed, or stripped from the core, for evanescent wave coupling, of the sensing polymer 1724 may dissect the core completely. Moreover, in this embodiment, the signal that is generated within the sensor 1714 can be returned and/or transmitted via a separate energy guide to the sensor assembly 216 (illustrated in FIG. 2A).

Yet alternatively, the transmission facilitator 1774 can have another suitable design.

FIG. 18 is a simplified schematic illustration of yet another embodiment of the sensor 1814 that can be included as part of the analyte sensing system 210 (illustrated in FIG. 2A), with the sensor 1814 yet again being configured without the specific inclusion of an energy guide. In particular, as illustrated in FIG. 18, the sensor 1814 again includes a sheath 1820 that defines at least a part of a reaction chamber 1822 that retains a transduction matrix 1826 therein, and a sensing polymer 1824 that is positioned within the sheath 1820. In this embodiment, the sensing polymer 1824 can again be provided simply as a piece of sensing material that is inserted into the sheath 1820.

However, in this embodiment, the sensor 1814 can be further modified to enable the sheath 1820 to function in a hybrid-type capacity, thus performing the functions of both the traditional sheath as well as the traditional energy guide. In some non-exclusive embodiments, in order to enable the sheath 1820 to perform this hybrid functionality, a portion of the sheath 1820 can be filled with an index material 1876. In certain embodiments, the index material 1876 can be a high refractive index material. Alternatively, the index material 1876 can have another suitable design, and/or the hybrid-type capacity for the sheath 1820 can be accomplished in another suitable manner.

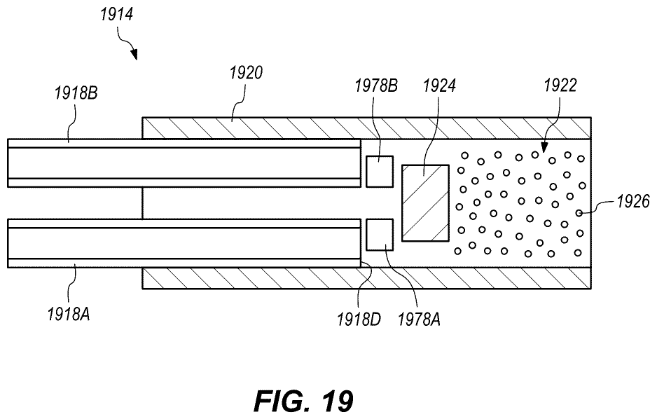

FIG. 19 is a simplified schematic illustration of still yet another embodiment of the sensor 1914 that can be included as part of the analyte sensing system 210 (illustrated in FIG. 2A), with the sensor 1914 including multiple energy guides, such as a first energy guide 1918A and a second energy guide 1918B.

As with the previous embodiments, the sensor 1914 can again include a sheath 1920 that defines at least a part of a reaction chamber 1922 that retains a transduction matrix 1926 therein, and a sensing polymer 1924 that is positioned within the sheath 1920.

However, as noted, in this embodiment, the sensor 1914 includes both the first energy guide 1918A and the second energy guide 1918B, which can each be configured to guide energy from the energy source 228 (illustrated in FIG. 2A) toward the sensing polymer 1924 and/or the transduction matrix 1926, and/or can be configured to transmit the signal from the sensor 1914 back toward the sensor assembly 216 (illustrated in FIG. 2A).

In some embodiments, the sensor 1914 can further include a first optical assembly 1978A that coupled to and/or positioned near a guide distal end 1918D of the first energy guide 1918A, and a second optical assembly 1978B that coupled to and/or positioned near a guide distal end 1918D of the second energy guide 1918B.

The first optical assembly 1978A can have any number and design of optical elements for purposes of directing and/or focusing the energy from the energy source 228 toward the sensing polymer 1924 and/or to direct returning energy or signal from the sensing polymer 1924 back to the first energy guide 1918A. More particularly, in certain embodiments, the first optical assembly 1978A can include one or more mirrors, lenses, prisms, gratings, holographic optical elements, computer generated holograms or other optical elements to focus, fold, bend, steer or otherwise redirect the energy to and from the first energy guide 1918A. In some embodiments, the first optical assembly 1978A can further include a collimator (such as a collimating lens) for collimating the energy that is directed to and from the first energy guide 1918A.

Similarly, the second optical assembly 1978B can have any number and design of optical elements for purposes of directing and/or focusing the energy from the energy source 228 toward the sensing polymer 1924 and/or to direct returning energy or signal from the sensing polymer 1924 back to the second energy guide 1918B. More particularly, in certain embodiments, the second optical assembly 1978B can include one or more mirrors, lenses, prisms, gratings, holographic optical elements, computer generated holograms, or other optical elements to focus, fold, bend, steer, collimate or otherwise redirect the energy to and from the second energy guide 1918B. In some embodiments, the second optical assembly 1978B can further include a collimator (such as a collimating lens) for collimating the energy that is directed to and from the first energy guide 1918B.

In summary, the various embodiments of the multi-channel, optical-based sensor described herein provide various advantages and/or benefits in comparison to the more traditional, widely used single channel design that has been used over the past 15+ years. As described in many embodiments, the foundation of the sensor design of the present invention is an oxygen sensor, which allows for another level of quality assurance by providing near real time self-calibration and site health monitoring. The opto-enzymatic design is also nearly impervious to most medication reactions, and many antioxidant foods can be adjusted for with a built-in oxygen reference channel.

Moreover, the present invention provides various advantages and/or benefits relative to electrochemical sensors, which suffer from various issues as noted above. More specifically, the multi-channel, optical-based sensor design incorporated within the present invention provides advantages and/or benefits such as (1) Minimization of Low Limit of Detection (LOD) and Non-Specific Adsorption, (2) Improvement in Reproducibility and Stability, and (3) Minimization of Biofouling, Fibrous Encapsulation, Inflammation, and Loss of Host Vasculature.

More particularly, low limit of detection (LOD) and non-specific adsorption can be minimized by implementing optical measurement of oxygen consumption using an oxygen sensitive polymer to decouple from interfering electroactive components such as hydrogen peroxide and even some pharmaceuticals such as acetaminophen or even vitamin C. This reduces non-specific interferences as well as improving the lower limit of detection. The sensor can also be tuned for a wide dynamic range to improve sensitivity and the lower detection limit. In particular, as described in various embodiments, the sensitivity of the sensor can be tuned for each channel by utilizing an annular-shaped or cylindrical-shaped oxygen controlling sheath where the oxygen permeability is controlled by design based on factors such as the material selection, sheath wall thickness, etc.

Moreover, the sensing polymer, such as the OSP in many embodiments, can be tuned for specific oxygen sensitivity and dynamic range by dye selection, polymer, co-polymer ratios, blends of miscible materials, composites of materials that may moderate oxygen diffusion, material selection, changing of cure parameters such as initiator amount, cure temperature, time, etc. to affect the molecular weight and/or the oxygen permeability of the polymerized material, ratios regarding dye concentration for amplitude, and hydrophobicity in order to avoid quenching by water or dissolved interfering ions or quenchers in the environment. The transduction matrix cross linker and/or average molecular weight can also be utilized to control the analyte (glucose, lactate, ketones, etc.) permeability, with the chamber length of the reaction chamber within which the transduction matrix is retained helping to control the range of sensitivity for a given sensor configuration. As noted above, achieving a low level of detection is crucial for detecting low concentrations of analytes, which is often required for early disease diagnosis; and suppressing the non-specific adsorption of interfering species is necessary to avoid false readings and to maintain sensor accuracy.

Additionally, reproducibility and stability can be improved by consistent fiber manufacturing using technology from the telecom industry, as well as consistent tube or sheath size that defines the transduction matrix dimensions. amplitude variation in optical pathways, light sources, and detectors. As noted above, ensuring consistent performance over time and in different conditions is challenging especially in the complex environment of the body.

Further, biofouling, fibrous encapsulation, inflammation, and loss of host vasculature can be minimized by keeping the sensor design smaller, and/or coating the sensor with bio-compliant materials. The effects of these can also be minimized by using a reference channel for monitoring oxygen, and/or temperature, and/or pH, to verify site health, namely oxygen availability, which is an indication of site health. The sensor can then send error messages when the readings should not be relied on and/or indicate when the sensor is encapsulated. Redundancy checks with channels that have overlapping sensitivity can also be employed within the sensors described herein. In particular, in many embodiments of the multi-channel sensors, the reference channel can be utilized to measure tissue oxygen that can monitor site health that would cause inaccurate sensor performance. The sensors can also utilize the coating on the sheath, which can be formed from a bio-compliant material or surface treatment, to effectively create an implantable biosensor that is designed to improve longevity when the analyte sensing system and/or the sensor are used in-vivo. As noted above, the accumulation of biological material on the sensor surface can interfere with sensor function and lead to inaccurate readings; the body's response to a foreign object can lead to encapsulation of the sensor, thereby impairing its function; and the body's immune response can cause inflammation around the sensor, affecting its accuracy and leading to potential complications. Thus, the use of such a coating on the sheath can greatly improve the long-term functionality, accuracy and reliability of the sensor. Such a coating can include a drug eluting component, releasing a drug (one example heparin) or steroid or hormone (some example such as dexamethasone acetate, Methylprednisolone, dexamethasone phosphate, Prednisolone, Hydrocortisone) to suppress the immune response in various alternative, non-exclusive embodiments.

It is understood that although a number of different embodiments of the analyte sensing system 210 and/or the sensor 214 have been illustrated and described herein, one or more features of any one embodiment can be combined with one or more features of one or more of the other embodiments, provided that such combination satisfies the intent of the present invention.

While a number of exemplary aspects and embodiments of the analyte sensing system 210 and/or the sensor 214 have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

1. An analyte sensing system for sensing an analyte within blood, bodily fluid, or tissue of a patient, the analyte sensing system comprising:
a sensor assembly including an energy source that generates energy; and
a sensor including (i) an energy guide that receives the energy from the energy source, the energy guide including a guide distal end (ii) a sheath that is coupled to the energy guide near the guide distal end, the sheath being substantially cylindrical-shaped to define at least a portion of a reaction chamber therewithin that extends distally away from the guide distal end, the sheath being oxygen permeable, and the sheath being impermeable to the analyte being sensed, the sheath having a sheath distal end, (iii) a sensing polymer that is positioned near the guide distal end of the energy guide, the energy guide guiding the energy from the energy source toward the sensing polymer, the sensing polymer being configured to sense one of oxygen and the analyte, the sensing polymer defining a chamber proximal end of the reaction chamber, and (iv) a transduction matrix that is retained substantially within the reaction chamber;
wherein the oxygen that permeates through the sheath and into the transduction matrix that is retained within the reaction chamber follows a first diffusion path within the transduction matrix; and
wherein the analyte permeates into the transduction matrix that is retained within the reaction chamber through the sheath distal end, the analyte following a second diffusion path within the transduction matrix that is different than the first diffusion path.

2. The analyte sensing system of claim 1 wherein the sensing polymer is coated onto the guide distal end of the energy guide.

3. (canceled)

4. The analyte sensing system of claim 1 wherein the sensing polymer is hydrophobic; and wherein the transduction matrix is hydrophilic.

5. The analyte sensing system of claim 1 wherein the sensing polymer is an oxygen sensing polymer that is configured to sense the oxygen within the transduction matrix.

6. The analyte sensing system of claim 1 wherein the sensing polymer is configured to directly sense the analyte within the transduction matrix.

7. The analyte sensing system of claim 1 wherein the sheath is formed via one of a three-dimensional extrusion technique and a three-dimensional molding technique.

8. The analyte sensing system of claim 1 wherein the sheath has a concentric unibody design.

9. The analyte sensing system of claim 1 wherein the substantially cylindrical shape of the sheath defines a chamber diameter of the reaction chamber, the chamber diameter being between approximately 100 nanometers and 500 micrometers.

10. The analyte sensing system of claim 1 wherein the sheath is formed from one or more of fluorinated ethylene propylene (FEP), paraformaldehyde (PFA), polytetrafluoroethylene (PTFE), polyimide, polyether block amide (PEBA), polyvinylchloride (PVC), polydimethylsiloxane, polyurethane, polyethylene, polycarbonate, poly(1-trimethylsilyl-1-propyne) (PTMSP), ethylene vinyl alcohol (EVOH), sulfonated tetrafluoroethylene-based fluoropolymer-copolymer, and modified cellulose.

11. The analyte sensing system of claim 10 wherein the sheath is formed at least partially from fluorinated ethylene propylene (FEP).

12. The analyte sensing system of claim 1 wherein the transduction matrix is comprised of a hydrogel and one or more enzymes that are configured to react with the oxygen and the analyte; and wherein a reaction between the one or more enzymes with the oxygen and the analyte consumes at least a portion of the oxygen and the analyte that is present within the transduction matrix.

13. The analyte sensing system of claim 12 wherein the transduction matrix further includes a catalyst that is configured to initiate the reaction between the oxygen and the analyte.

14. The analyte sensing system of claim 1 wherein the sheath has a wall thickness that impacts the permeability of the sheath to oxygen; and wherein the wall thickness of the sheath is between approximately 10 micrometers and 400 micrometers.

15. The analyte sensing system of claim 1 wherein the sheath has an oxygen permeability of between approximately five (cm³ mm/m²·day·bar)/mm and 60,000 (cm³ mm/m²·day·bar)/mm.

16. The analyte sensing system of claim 1 wherein the sensor further includes a buffer layer that is positioned about the energy guide, the buffer layer being formed from one or more polymeric materials.

17. The analyte sensing system of claim 1 wherein the energy source is a light source that generates light energy; and wherein the energy guide is an optical fiber.

18. The analyte sensing system of claim 1 wherein the sensor is a first sensor channel; and wherein the analyte sensing system further includes a second sensing channel that includes (i) a second energy guide that receives the energy from the energy source, the second energy guide including a second guide distal end, (ii) a second sheath that is coupled to the second energy guide near the second guide distal end, the second sheath defining at least a portion of a second reaction chamber, the second sheath being oxygen permeable, and the second sheath being impermeable to the analyte being sensed, (iii) a second sensing polymer that is positioned near the second guide distal end of the second energy guide, the second energy guide guiding the energy from the energy source toward the second sensing polymer, the second sensing polymer being configured to sense one of the oxygen and the analyte, and (iv) a second transduction matrix that is retained substantially within the second reaction chamber.

19. An analyte sensing system for sensing an analyte within blood, bodily fluid, or tissue of a patient, the analyte sensing system comprising:
a sensor assembly including an energy source that generates energy; and
a sensor including (i) an energy guide that receives the energy from the energy source, the energy guide including a guide distal end (ii) a sheath that is coupled to the energy guide near the guide distal end, the sheath being substantially cylindrical-shaped to define at least a portion of a reaction chamber therewithin that extends distally away from the guide distal end, the sheath being oxygen permeable, and the sheath being impermeable to the analyte being sensed, the sheath having a sheath distal end, (iii) a sensing polymer that is positioned near the guide distal end of the energy guide, the energy guide guiding the energy from the energy source toward the sensing polymer, the sensing polymer being configured to sense one of oxygen and the analyte, the sensing polymer defining a chamber proximal end of the reaction chamber, and (iv) a transduction matrix that is retained substantially within the reaction chamber;
wherein the oxygen that permeates through the sheath and into the transduction matrix that is retained within the reaction chamber follows a first diffusion path within the transduction matrix;
wherein the analyte permeates into the transduction matrix that is retained within the reaction chamber through the sheath distal end, the analyte following a second diffusion path within the transduction matrix that is different than the first diffusion path; and
wherein the sensor is a first sensor channel; and wherein the analyte sensing system further includes a reference channel that includes (i) a reference energy guide that receives the energy from the energy source, the reference energy guide including a guide distal end, (ii) a reference sheath that is coupled to the reference energy guide near the guide distal end, and (iii) a reference sensing polymer that is configured to sense oxygen from within the blood, bodily fluid or tissue of the patient to set a baseline level of oxygen within the blood, bodily fluid or tissue of the patient.

20. An analyte sensing system for sensing an analyte within blood, bodily fluid, or tissue of a patient, the analyte sensing system comprising:
a sensor assembly including an energy source that generates energy; and
a sensor including (i) an energy guide that receives the energy from the energy source, the energy guide including a guide distal end, (ii) a sheath that is coupled to the energy guide near the guide distal end, the sheath having a concentric unibody design that is substantially cylindrical-shaped to define at least a portion of a reaction chamber therewithin that extends distally away from the guide distal end, the sheath being oxygen permeable, and the sheath being impermeable to the analyte being sensed, the sheath having a sheath distal end, (iii) a hydrophobic oxygen sensing polymer that is coated onto the guide distal end of the energy guide, the oxygen sensing polymer being configured to sense oxygen within the reaction chamber, the oxygen sensing polymer defining a chamber proximal end of the reaction chamber, the energy guide guiding the energy from the energy source toward the oxygen sensing polymer, and (iv) a hydrophilic transduction matrix that is retained substantially within the reaction chamber, the transduction matrix including a hydrogel and one or more enzymes that are configured to react with the oxygen and the analyte;
wherein the oxygen that permeates through the sheath and into the transduction matrix that is retained within the reaction chamber follows a first diffusion path within the transduction matrix;
wherein the analyte permeates into the transduction matrix that is retained within the reaction chamber through the sheath distal end of the sheath, the analyte following a second diffusion path within the transduction matrix that is different than the first diffusion path; and
wherein a reaction between the one or more enzymes with the oxygen and the analyte consumes at least a portion of the oxygen and the analyte that is present within the transduction matrix.

21. The analyte sensing system of claim 18 wherein the reaction chamber further includes a chamber distal end; wherein the second reaction chamber includes a second chamber proximal end and a second chamber distal end; and
wherein at least one of (i) the chamber proximal end is staggered relative to the second chamber proximal end, and (ii) the chamber distal end is staggered relative to the second chamber distal end.
